(19) United States
(12) Patent Application Publication
(10) Pub. No.: US 2006/0128014 A1
(43) Pub. Date: Jun. 15, 2006

(54) Compositions and methods for culturing stem cells

(76) Inventors: Johan Haggblad, Stockholm (SE); Carolyn Horrocks, Stockholm (SE); Katarina Jansson, Johanneshov (SE); Harriet Ronnholm, Trangsund (SE)

Correspondence Address: JONES DAY, 222 EAST 41ST ST, NEW YORK, NY, 10017, US

(21) Appl. No.: 10/525,930

(22) PCT Filed: Aug. 26, 2003
(86) PCT No.: PCT/IB03/04388

§ 371 Date: Jan. 5, 2006

Related U.S. Application Data
(60) Provisional application No. 60/406,688, filed on Aug. 26, 2002.

(51) Int. Cl. C12N 5/08

(52) U.S. Cl. 435/368

(57) ABSTRACT

The present invention provides methods of culturing, propagating, treating, and maintaining stem cells in the presence of a phosphate mimic. In particular, the invention relates to the propagation of stem cells in vitro, the formation of neurospheres in vitro, and to tissue culture of stem cells in the presence of a phosphate mimic. The invention further relates to the treatment of neurodegenerative disorders.

### RELATED APPLICATIONS

This application claims benefit under 35 U.S.C. § 119(e) of U.S. Provisional Application No. 60/406,688, filed on Aug. 26, 2002, which is incorporated by reference herein in its entirety.

### 1. FIELD OF THE INVENTION

The present invention relates to a method of culturing and maintaining stem cells and progenitor cells. In particular, the invention relates to the propagation of stem cells and progenitor cells in vitro, the formation of neurospheres in vitro, and to tissue culture of stem cells and progenitor cells in the presence of a stem and/or progenitor cell growth-modulating agent. The stem and/or progenitor cell growth-modulating agent can be a phosphate mimic. The invention further relates to the treatment of neurodegenerative disorders.

### 2. BACKGROUND OF THE INVENTION

Traditionally growth of stem cells and progenitor cells is triggered by administration of various protein growth factors. This treatment, be it in vitro or in vivo, is expensive and complex due to the protein nature of the factors. Here is described a method that circumvents and/or reduces the use of protein growth factors for the purpose of treatment of stem cell derived disease and for production of stem cells and cells derived from stem cells.

The activities of protein tyrosine phosphatases (PTPases), and other enzymes that bind phosphate groups, are inhibited by phosphate bio-isosteres like vanadate and derivatives thereof. Some of these enzymes, especially the PTPases (e.g., but not limited to PTP1B, CD45, PTP1C, PTP-alpha, LAR and HePTP) are involved in the control of growth of cells. The proliferation of neural stem cells is controlled by, e.g., protein growth factors acting on growth factor receptors and cognate pathways.

Phosphorylation of proteins regulates enzyme activities and other protein function. Signal transduction from cell surface to cell nucleus (and the reverse) is in part mediated by protein phosphatases. Some of such signal transduction mediates proliferation of cells. Proliferation of cells is important for homeostasis mechanisms in any organism and is also a prerequisite for expansion and maintenance of cultured cells and tissues.

Phosphorylation/dephosphorylation of non-proteinaceous bio-molecules also constitute important metabolic- and control events in various pathways and cycles, such as, e.g., the glycolysis and the citric acid cycle, and the maintenance of cell membrane potential. It is very likely such mechanisms also influence cell proliferation.

The term “signal transduction” is a collective term used to define all cellular processes that follow stimulation of a given cell or tissue and result in a response by the cell to the stimulus. Examples of signal transduction include but are not in any way limited to cellular events that are induced by polypeptide hormones and growth factors (e.g. insulin, insulin-like growth factors I and II, growth hormone, epidermal growth factor, platelet-derived growth factor), cytokines (e.g. interleukines), extracellular matrix components, and cell-cell interactions.

Phosphotyrosine recognition units are defined as areas or domains of proteins or glycoproteins that have affinity for molecules containing phosphorylated tyrosine residues (phosphotyrosine; pTyr). Examples of pTyr recognition units include but are not in any way limited to: PTPases, SH2 domains and PTB domains.

PTPases are defined as enzymes with the capacity to dephosphorylate pTyr-containing proteins, including, e.g., glycoproteins. Examples of PTPases include but are not in any way limited to: intracellular PTPases (e.g. PTP-1B, TC-PTP, PTP-1C, PTP-1D, PTP-D1, PTP-D2), receptor-type PTPases (e.g., PTPalpha, PTPepsilon, PTPbeta, PTPgamma, CD45, PTPkappa, PTPmu), dual specificity phosphatases (e.g. VH1, VHR, cdc25) and other PTPases such as LAR, SHP-1, SHP-2, PTP-1H, PTPMEGI, PTP-PEST, PTP.zeta., PTPS31, IA-2 and HePTP and the like.

Vanadate is tolerated in humans and the effect of vanadate has been tested with positive results in patients suffering from diabetes. For example, vanadyl sulfate was given orally to subjects with diabetes at a dose of 25, 50, or 100 mg vanadium (V) daily (Goldfine et al., Metabolism 49 (2000) 1-12), Goldfine A B, Simonson D C, Folli F, Patti M E, Kahn C R Mol Cell Biochem 1995 Dec. 6-20; 153(1-2):217-31). The most common adverse effect of oral NaVO3 was mild gastrointestinal intolerance (Goldfine A B, Simonson D C, Folli F, Patti M E, Kahn C R, J Clin Endocrinol Metab 1995 November; 80(11):3311-20). Vanadate also reduces blood glucose levels in experimental animals (eg Ding W, Hasegawa T, Hosaka H, Peng D, Takahashi K, Seko Y, Biol Trace Elem Res 2001 May; 80(2):159-74)

In experimental animals vanadate was shown to be neuroprotective. In transient forebrain ischemia, sodium orthovanadate as well as insulinlike growth factor-1 (IGF-1) rescued cells from delayed neuronal death in the hippocampal CA1 region. (Kawano T, Fukunaga K, Takeuchi Y, Morioka M, Yano S, Hamada J, Ushio Y, Miyamoto E, J Cereb Blood Flow Metab 2001 November; 21(11):1268-80).

### 3. SUMMARY OF THE INVENTION

The invention provides a method for culturing a stem cell, said method comprising propagating the stem cell in tissue culture medium comprising one or more agents selected from the group consisting of an inhibitor of a PTPase, a modulator of an enzyme with one or more phosphate binding sites, a phosphohydrolase, a pyrophosphatase, an alkaline phosphatase, an acid phosphatase, and a modulator of a protein with one or more pTyr recognition unit. In specific embodiments, the pTyr recognition unit is a SH2 domain. In specific embodiments, the enzyme with one or more phosphate binding sites is a glucose-6-phosphate dehydrogenase, a fructose-2,6-bisphosphatase, a phosphoglucomutase, a Mg²⁺ dependent ATPase, a plasma membrane Ca²⁺ ATPases, an endoplamic reticulum Ca²⁺-ATPases, a P-glycoprotein ATPase activity, a Mg²⁺-dependent vanadate-sensitive GS-conjugate export ATPase, a MRP/GS-X pump, or a Na⁺, K⁺-ATPase.

The invention further provides a method for culturing a stem cell, said method comprising propagating the stem cell in tissue culture medium comprising a phosphate mimic or a stem and/or progenitor cell growth-modulating agent.

The invention further provides a method for culturing a stem cell, said method comprising incubating the stem cells in tissue culture medium comprising a phosphate mimic or a stem and/or progenitor cell growth-modulating agent, wherein the stem cell undergoes self-renewal.

The invention further provides a method for culturing a stem cell, said method comprising incubating the stem cell in tissue culture medium comprising between 1 μM and 100 μM vanadate.

The invention further provides a method for culturing a stem cell, said method comprising incubating the stem cell in tissue culture medium comprising a phosphate mimic or a stem and/or progenitor cell growth-modulating agent, wherein the culture medium is not supplemented with exogenously added growth factor.

The invention further provides a method for culturing a stem cell, said method comprising incubating the stem cell in tissue culture medium comprising a phosphate mimic or a stem and/or progenitor cell growth-modulating agent, wherein the incubating step elevates intracellular ATP levels in the neural stem cell by at least 25% compared to intracellular ATP levels in the stem cell incubated without phosphate mimic or a stem and/or progenitor cell growth-modulating agent under otherwise same conditions. In specific embodiments, the incubating step elevates intracellular ATP levels in the neural stem cell by at least 50% compared to intracellular ATP levels in the stem cell incubated without phosphate mimic or a stem and/or progenitor cell growth-modulating agent under otherwise same conditions. In specific embodiments, the incubating step elevates intracellular ATP levels in the neural stem cell by at least 100% compared to intracellular ATP levels in the stem cell incubated without phosphate mimic or a stem and/or progenitor cell growth-modulating agent under otherwise same conditions. In specific embodiments, the incubating step elevates intracellular ATP levels in the neural stem cell by at least 200% compared to intracellular ATP levels in the stem cell incubated without phosphate mimic or a stem and/or progenitor cell growth-modulating agent under otherwise same conditions. In specific embodiments, the incubating step elevates intracellular ATP levels in the neural stem cell by at least 300% compared to intracellular ATP levels in the stem cell incubated without phosphate mimic or a stem and/or progenitor cell growth-modulating agent under otherwise same conditions. In specific embodiments, the incubating step elevates intracellular ATP levels in the neural stem cell by at least 400% compared to intracellular ATP levels in the stem cell incubated without phosphate mimic or a stem and/or progenitor cell growth-modulating agent under otherwise same conditions. In specific embodiments, the incubating step elevates intracellular ATP levels in the neural stem cell by at least 500% compared to intracellular ATP levels in the stem cell incubated without phosphate mimic or a stem and/or progenitor cell growth-modulating agent under otherwise same conditions.

In certain embodiments, the invention further provides a method for culturing a population of stem cells or progenitor cells, respectively, said method comprising incubating the stem cells or progenitor cells, respectively, in tissue culture medium comprising a phosphate mimic or a stem and/or progenitor cell growth-modulating agent, wherein the incubating step elevates ATP levels per a certain volume of culture, e.g., per 1 ml of culture, by at least 25%, 50%, 100%, 200%, 300% or at least 400% compared to ATP levels per the certain volume of cell culture, e.g., per ml of culture, without phosphate mimic or a stem and/or progenitor cell growth-modulating agent under otherwise same conditions.

In certain embodiments, the invention further provides a method for culturing a population of stem cells or progenitor cells, respectively, said method comprising incubating the stem cells or progenitor cells, respectively, in tissue culture medium comprising a phosphate mimic or a stem and/or progenitor cell growth-modulating agent, wherein the incubating step elevates the total ATP level of the plurality of cells per a certain volume of culture, e.g., per 1 ml of culture, by at least 25%, 50%, 100%, 200%, 300% or at least 400% compared to the total ATP level of the plurality of cells per the certain volume of cell culture without phosphate mimic or a stem and/or progenitor cell growth-modulating agent under otherwise same conditions.

The invention provides a method of identifying a candidate gene that is modulated in a stem cell by treatment with a phosphate mimic or a stem and/or progenitor cell growth-modulating agent, said method comprising the following steps: culturing one or more stem cell populations in the presence of one or more concentrations of the phosphate mimic or a stem and/or progenitor cell growth-modulating agent for a 72- to 96-hour period; (b) culturing one or more stem cell populations without the phosphate mimic or a stem and/or progenitor cell growth-modulating agent to the culture for a 72- to 96-hour period; and (c) identifying any gene that is differentially expressed between the culturing steps (a) and (b) in the stem cell, wherein a gene that is differentially expressed between the culturing steps (a) and (b) is the candidate gene that is modulated in a stem cell by treatment with the phosphate mimic or a stem and/or progenitor cell growth-modulating agent.

In certain embodiments of the invention, the stem cell is a fetal neural stem cell. In certain embodiments of the invention, the stem cell is an adult neural stem cell. In certain embodiments of the invention, the stem cell is an embryonal stem cell. In certain embodiments of the invention, the stem cell is an ependymal neural CNS stem cells.

The invention further provides a method for culturing a neural stem cell, said method comprising incubating the neural stem cell in tissue culture medium comprising a phosphate mimic or a stem and/or progenitor cell growth-modulating agent, wherein the incubating step increases formation of neurospheres from the neural stem cell by at least 25% compared to the formation of neurospheres from the stem cell incubated without phosphate mimic or a stem and/or progenitor cell growth-modulating agent under otherwise same conditions. In certain embodiments, the incubating step increases formation of neurospheres from the neural stem cell by at least 50% compared to the formation of neurospheres from the stem cell incubated without phosphate mimic or a stem and/or progenitor cell growth-modulating agent under otherwise same conditions. In certain embodiments, the incubating step increases formation of neurospheres from the neural stem cell by at least 100% compared to the formation of neurospheres from the stem cell incubated without phosphate mimic or a stem and/or progenitor cell growth-modulating agent under otherwise same conditions. In certain embodiments, the incubating step increases formation of neurospheres from the neural stem cell by at least 200% compared to the formation of neurospheres from the stem cell incubated without phosphate mimic or a stem and/or progenitor cell growth-modulating agent under otherwise same conditions. In certain embodiments, the incubating step increases formation of neurospheres from the neural stem cell by at least 300% compared to the formation of neurospheres from the stem cell incubated without phosphate mimic or a stem and/or progenitor cell growth-modulating agent under otherwise same conditions. In certain embodiments, the incubating step increases formation of neurospheres from the neural stem cell by at least 400% compared to the formation of neurospheres from the stem cell incubated without phosphate mimic or a stem and/or progenitor cell growth-modulating agent under otherwise same conditions. In certain embodiments, the incubating step increases formation of neurospheres from the neural stem cell by at least 500% compared to the formation of neurospheres from the stem cell incubated without phosphate mimic or a stem and/or progenitor cell growth-modulating agent under otherwise same conditions. In certain embodiments, the neural stem cell is a fetal neural stem cell. In certain embodiments, the neural stem cell is an adult neural stem cell.

The invention further provides a stem cell, wherein the cultured stem cell has been generated by a method of the invention.

The invention further provides a cultured neural stem cell, wherein the cultured neural stem cell has been generated by a method of the invention.

The invention further provides a method for culturing a progenitor cell, said method comprising propagating the progenitor cell in tissue culture medium comprising a phosphate mimic or a stem and/or progenitor cell growth-modulating agent.

The invention further provides a for culturing a progenitor cell, said method comprising incubating the progenitor cell in tissue culture medium comprising a phosphate mimic or a stem and/or progenitor cell growth-modulating agent, wherein the progenitor cell undergoes self-renewal.

The invention further provides a method for culturing a progenitor cell, said method comprising incubating the progenitor cell in tissue culture medium comprising a phosphate mimic or a stem and/or progenitor cell growth-modulating agent, wherein the culture medium is not supplemented with exogenously added growth factor.

The invention further provides a method for culturing a progenitor cell, said method comprising incubating the progenitor cell in tissue culture medium comprising a phosphate mimic or a stem and/or progenitor cell growth-modulating agent, wherein the incubating step increases intracellular ATP levels in the progenitor cell by at least 25% compared to intracellular ATP levels in the progenitor cell incubated without phosphate mimic or a stem and/or progenitor cell growth-modulating agent under otherwise same conditions. In specific embodiments, the incubating step increases intracellular ATP levels in the progenitor cell by at least 50% compared to intracellular ATP levels in the progenitor cell incubated without phosphate mimic or a stem and/or progenitor cell growth-modulating agent under otherwise same conditions. In specific embodiments, the incubating step increases intracellular ATP levels in the progenitor cell by at least 100% compared to intracellular ATP levels in the progenitor cell incubated without phosphate mimic or a stem and/or progenitor cell growth-modulating agent under otherwise same conditions. In specific embodiments, the incubating step increases intracellular ATP levels in the progenitor cell by at least 200% compared to intracellular ATP levels in the progenitor cell incubated without phosphate mimic or a stem and/or progenitor cell growth-modulating agent under otherwise same conditions. In specific embodiments, the incubating step increases intracellular ATP levels in the progenitor cell by at least 300% compared to intracellular ATP levels in the progenitor cell incubated without phosphate mimic or a stem and/or progenitor cell growth-modulating agent under otherwise same conditions. In specific embodiments, the incubating step increases intracellular ATP levels in the progenitor cell by at least 400% compared to intracellular ATP levels in the progenitor cell incubated without phosphate mimic or a stem and/or progenitor cell growth-modulating agent under otherwise same conditions. In specific embodiments, the incubating step increases intracellular ATP levels in the progenitor cell by at least 500% compared to intracellular ATP levels in the progenitor cell incubated without phosphate mimic or a stem and/or progenitor cell growth-modulating agent under otherwise same conditions.

The invention further provides a method for culturing a progenitor cell, said method comprising incubating the neural progenitor cell in tissue culture medium comprising a phosphate mimic or a stem and/or progenitor cell growth-modulating agent, wherein the progenitor cell is at least 25% more proliferative than the neural progenitor cell incubated without a phosphate mimic or a stem and/or progenitor cell growth-modulating agent under otherwise same conditions, and wherein the proliferation is measured by a method comprising: (a) culturing one or more progenitor cell populations in the presence of one or more concentrations of the phosphate mimic or a stem and/or progenitor cell growth-modulating agent for a 72- to 96-hour period; (b) culturing one or more progenitor cell populations without the phosphate mimic or a stem and/or progenitor cell growth-modulating agent to the culture for a 72- to 96-hour period; and (c) determining the number of viable progenitor cells at the end of the 72- to 96-hour period in the stem cell populations of step (a) and (b), respectively, and wherein the culturing steps (a) and (b) are conducted under otherwise the same conditions. In specific embodiments, the neural progenitor cell is at least 50% more proliferative than the progenitor cell incubated without a phosphate mimic or a stem and/or progenitor cell growth-modulating agent under otherwise same conditions. In specific embodiments, the progenitor cell is at least 100% more proliferative than the progenitor cell incubated without a phosphate mimic or a stem and/or progenitor cell growth-modulating agent under otherwise same conditions. In specific embodiments, the progenitor cell is at least 200% more proliferative than the progenitor cell incubated without a phosphate mimic or a stem and/or progenitor cell growth-modulating agent under otherwise same conditions. In specific embodiments, the progenitor cell is at least 300% more proliferative than the progenitor cell incubated without a phosphate mimic or a stem and/or progenitor cell growth-modulating agent under otherwise same conditions. In specific embodiments, the progenitor cell is at least 400% more proliferative than the progenitor cell incubated without a phosphate mimic or a stem and/or progenitor cell growth-modulating agent under otherwise same conditions. In specific embodiments, the progenitor cell is at least 500% more proliferative than the progenitor cell incubated without a phosphate mimic or a stem and/or progenitor cell growth-modulating agent under otherwise same conditions.

In certain embodiments of the invention, the progenitor cell is a neural progenitor cell.

The invention provides a method for culturing a neural progenitor cell, said method comprising incubating the neural progenitor cell in tissue culture medium comprising a phosphate mimic or a stem and/or progenitor cell growth-modulating agent, wherein the incubating step increases formation of neurospheres from the neural progenitor cell by at least 25% compared to the formation of neurospheres from the neural progenitor cell incubated without phosphate mimic or a stem and/or progenitor cell growth-modulating agent under otherwise same conditions. In specific embodiments, the incubating step increases formation of neurospheres from the neural progenitor cell by at least 50% compared to the formation of neurospheres from the neural progenitor cell incubated without phosphate mimic or a stem and/or progenitor cell growth-modulating agent under otherwise same conditions. In specific embodiments, the incubating step increases formation of neurospheres from the neural progenitor cell by at least 100% compared to the formation of neurospheres from the neural progenitor cell incubated without phosphate mimic or a stem and/or progenitor cell growth-modulating agent under otherwise same conditions. In specific embodiments, the incubating step increases formation of neurospheres from the neural progenitor cell by at least 200% compared to the formation of neurospheres from the neural progenitor cell incubated without phosphate mimic or a stem and/or progenitor cell growth-modulating agent under otherwise same conditions. In specific embodiments, the incubating step increases formation of neurospheres from the neural progenitor cell by at least 300% compared to the formation of neurospheres from the neural progenitor cell incubated without phosphate mimic or a stem and/or progenitor cell growth-modulating agent under otherwise same conditions. In specific embodiments, the incubating step increases formation of neurospheres from the neural progenitor cell by at least 400% compared to the formation of neurospheres from the neural progenitor cell incubated without phosphate mimic or a stem and/or progenitor cell growth-modulating agent under otherwise same conditions. In specific embodiments, the incubating step increases formation of neurospheres from the neural progenitor cell by at least 500% compared to the formation of neurospheres from the neural progenitor cell incubated without phosphate mimic or a stem and/or progenitor cell growth-modulating agent under otherwise same conditions.

The invention provides a cultured progenitor cell, wherein the cultured progenitor cell has been generated by a method of the invention.

In certain embodiments, the phosphate mimic is selected from the group consisting of a vanadium oxide, a derivative of a vanadium oxide, a polyoxometalate, a homopolyoxotungstate, a vanadium-substituted polyoxotungstate, an esterified derivative of 4-(fluoromethyl)phenyl phosphate, a homopolyoxoselenate, a vanadium-substituted polyoxoselenate, a homopolyoxomolybdate, a vanadium-substituted polyoxomolybdate, and a PTPase inhibitor.

In certain embodiments of the invention, the phosphate mimic is vanadate, orthovanadate, metavanadate, pervanadate, vanadate dimer, vanadate tetramer, vanadate pentamer, vanadate hexamer, vanadate heptamer, vanadate octamer, vanadate nonamer, vanadate decamer, vanadate polymer, vanadyl sulfate, bis(6, ethylpicolinato)(H(2)O)oxovanadium(IV) complex, bis(1-oxy-2-pyridinethiolato)oxovanadium(IV), bis(maltolato)oxovanadium (IV), bis(biguanidato)oxovanadium(IV), bis(N′N′-dimethylbiguanidato)oxovanadium(IV), bis(beta-phenethyl-biguanidato)oxovanadium(IV), peroxovanadate-nicotinic acid, aluminiofluoride, 4-(fluoromethyl)phenyl phosphate, tungstate, selenate, molybdate, Zn²⁺ or F⁻¹.

In certain specific embodiments, the phosphate mimic is vanadate. In certain specific embodiments, the concentration of vanadate in the culture medium is 1 μM. In certain specific embodiments, the concentration of vanadate in the culture medium is 10 μM. In certain specific embodiments, the concentration of vanadate in the culture medium is 50 μM. In certain specific embodiments, the concentration of vanadate in the culture medium is 100 μM. In certain specific embodiments, the concentration of vanadate in the culture medium is 500 μM. In certain specific embodiments, the concentration of vanadate in the culture medium is 1000 μM.

In certain embodiments, the culture medium further comprises an agent selected from the group consisting of a growth factor, a Receptor Tyrosine Kinase agonist, and a growth factor secretagogue. In certain embodiments, the culture medium further comprises an agent selected from the group consisting of an agonist of cAMP accumulation, a Ca²⁺-transient triggering factor, and an agonist of cGMP accumulation. In certain embodiments, the culture medium further comprises an agent selected from the group consisting of a GPCR agonist, a GPCR antagonist, an agonist of adenylate cyclase, an antagonist of phosphodiesterase, an antagonist of neurotransmitter uptake, a MAO inhibitor, a COMT inhibitor, a neuropeptide peptidase inhibitor, a Li-salt, an inhibitor of the sarcoplamic-reticulum Calcium-ATPase, an agonist of IP3, an agonist of IP3 receptor, a Calcium ionophore, a cell membrane depolarizing agent, an agonist of guanylate cyclase, an inhibitor of phosphodiesterase, a natriuretic peptide and a natriuretic peptide mimics. In certain embodiments, the cell does not substantially differentiate during the incubating step.

In certain embodiments, the culture medium for culturing stem cells comprises a phosphate mimic, wherein the culture medium has not been supplemented with any exogenously added growth factor, and wherein the culture medium supports the proliferation of the stem cell.

In certain embodiments, the culture medium for culturing stem cells comprises a phosphate mimic, wherein the culture medium comprises one or more growth factors in an amount that is not sufficient to support proliferation of the stem cell in the absence of a phosphate mimic, and wherein the culture medium supports the proliferation of the stem cell.

### 4. BRIEF DESCRIPTION OF THE DRAWINGS

FIG. 1. Concentration-response curve for vanadate added to cultured adult neural stem cells in supplemented BIT or B27. The intracellular ATP levels are plotted as a function of concentration of added vanadate, 0 corresponds to 1 micromolar, 1 to 10 micromolar etc.

FIG. 2. The effect of vanadate in the presence of EGF, in supplement BIT.

FIG. 3. The effect of vanadate in the presence of EGF, in supplement B27.

FIG. 4. The effect of vanadate in the presence of PACAP, in supplement BIT.

FIG. 5. The effect of vanadate in the presence of PACAP, in supplement B27.

FIG. 6. Sphere formation in the presence of vanadate and in combination with PACAP. Panel a) shows neurosphere formation in the presence of vanadate and panel b) shows neurosphere formation in the presence of vanadate and PACAP.

FIG. 7. Vanadate in combination with EGF. ATP-levels in cells cultured with 3 nM EGF and different concentrations of vanadate. Vanadate concentrations are shown on the x-axis in microM; ATP levels are shown in light units on the y-axis.

FIG. 8. Effects on human neural stem cells. Panel a) shows neurosphere formation of human neural stem cells in the presence of vanadate; panels b) and c) show reduced neurosphere formation from human neural stem cells in the presence of FGF-2 (panel b) and EGF and FGF-2 (c), respectively.

### ABBREVIATIONS AND CONVENTIONS

The term “stem and/or progenitor cell growth-modulating agent” as used herein refers to a substance selected from the group consisting of inhibitors of PTPases, modulators of activities of enzymes with phosphate binding sites, modulators of activities of proteins with pTyr recognition units, and phosphate mimics. Enzymes with phosphate binding sites are, e.g., glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases. pTyr recognition units include, but are not limited to, SH2 domains, PTPases, and PTB domains. In a preferred embodiment, the stem and/or progenitor cell growth-modulating agent is a modulator of activity of an enzyme with one or more phosphate binding sites, wherein the enzyme with one or more phosphate binding sites is selected from the group consisting of glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases. In a preferred embodiment, the stem and/or progenitor cell growth-modulating agent is a modulator of activity of a protein with one or more pTyr recognition units, wherein the one or more pTyr recognition units are selected from the group consisting of SH2 domains, PTPases and PTB domains. Examples of modulators of enzymes with phosphate binding sites are: rolipram as modulator of phosphodiesterase IV; sildenafil as modulator of phosphodiesterase V; ouabain as modulator of Na/K ATPase; aurintricarboxylic acid as modulator of phosphofructokinase; staurosporin as modulator of protein kinase C; genistein as modulator of protein-Tyr kinase; gleevec(imatinib) as modulator of protein-Tyr kinase; di(t-butyl)-1,4-quinone as modulator of Ca-ATPase; and thapsigargin as modulators of Ca-ATPase. Peptides preventing the binding reaction between the p-Tyr recognition unit and its binding partner can be used as modulators of proteins with p-Tyr recognition units.

The term “phosphate mimic” as used herein refers to a substance that is not a phospate but is structurally similar to a phosphate such that the phosphate mimic is capable of binding to a phosphate binding site. Such phosphate binding sites include, but are not limited to, the phosphate binding sites of glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases. The term “phosphate mimic” includes, but is not limited to, agents, that bind to a domain that binds to pTyr, such as a SH2 domain or a PTB domain. In certain embodiments, a phosphate mimic is a molecule that comprises a moiety that is structurally similar to a phosphate such that the phosphate mimic is capable of binding to a phosphate binding site. The term “phosphate mimic” as used in the context of the present application relates to compounds that are, inter alia, capable of inhibiting a PTPase. In specific embodiments, the Ki of the phosphate mimic in inhibiting a PTPase in is at least 0.1 nM, at least 0.5 nM, at least 1 mM, at least 5 mM, at least 10 mM, or at least 25 mM. In specific embodiments, the Ki of the phosphate mimic in inhibiting a PTPase is at most 0.1 nM, at most 0.5 nM, at most 1 mM, at most 5 mM, at most 10 mM, or at most 30 mM.

The term “stem cell” as used herein refers to a cell that (a) is capable of self-renewal; and (b) is a cell from which other types of cells can develop.

The term “progenitor cell” as used herein refers to a cell that (a) is not capable of self-renewal; and (b) is a cell from which other types of cells can develop.

The terms “cell proliferation” and “to proliferate” as used herein refer to the amplification of the cell by cell division.

The term “support” when applied to conditions under which cells are maintained, cultured, grown, proliferated, propagated or renewed, refers to conditions under which cells are capable of, respectively, being maintained, being cultured, growing, proliferating, propagating or renewing. Conditions can include cell culture media, concentrations of phosphate mimic, concentrations of stem and/or progenitor cell growth-modulating agent, or concentrations of growth factors. For example, a given cell culture media is said to “support” cell proliferation when a cell grown in said media is capable of proliferating.

As used herein, the term “isolated” when applied to a cell refers to a cell isolated from an animal, (e.g., a human, a rat, a mouse, etc.) and purified up to at least about 10%, such as 80%. Purity is measured by comparing the number of neural stem cells with the total number of cells. For example, an “80% pure” preparation of ependymal neural stem cells means that 80% of the cells in the preparation are ependymal neural stem cells.

The term “neural stem cells” relates to cells capable of generating aggregates of undifferentiated cells, so called neurospheres, under suitable conditions. The term “Ependymal cells” refers to any cell originating from the ependymal layer in the CNS ventricular system or the same cell type located elsewhere. In the present context, it is to be understood that among the features that characterize the ependymal neural stem cells according to the invention is the capability thereof to generate new stem cells, precursors, progenitor cells, neurons, astroglia or oligodendroglia. For a description of such cells and methods of isolating such cells, see copending and co-owned U.S. patent application Ser. No. 09/104,772, filed Jun. 25, 1998, entitled “Method of Isolating Ependymal Neural Stem Cells”, by Frisen et al., and Ser. No. 09/719,001, filed Jul. 12, 2001, entitled “Ependymal Neural Stem Cells and Methods for Their Isolation”, by Janson et al., which are incorporated by reference in their entireties herein. The term “adult” is used herein to differentiate the neural stem cells previously identified in embryos from the present ependymal neural stem cells of the invention obtained from post-natal mammals. Thus, adult stem cells are in essence non-embryonic stem cells.

“Modulation of cellular processes”, e.g., signal transduction, is defined as the capacity of compounds that are to be used with the methods of the invention to 1) either increase or decrease ongoing, normal or abnormal, signal transduction, 2) initiate normal signal transduction, and 3) initiate abnormal signal transduction.

“Modulation of the activity of molecules with pTyr recognition units” is defined as the capacity of compounds of the invention to 1) increase or decrease the activity of proteins or glycoproteins with pTyr recognition units (e.g. PTPases, SH2 domains or PTB domains) or to 2) decrease or increase the association of a pTyr-containing molecule with a protein or glycoprotein with pTyr recognition units either via a direct action on the pTyr recognition site or via an indirect mechanism. Examples of modulation of the activity of molecules with pTyr recognition units, which are not intended in any way limiting to the scope of the invention claimed, are: a) inhibition of PTPase activity leading to increased, decreased, normal, or abnormal signal transduction; b) stimulation of PTPase activity leading to increased, decreased, normal, or abnormal signal transduction; c) inhibition of binding of SH2 domains or PTB domains to proteins or glycoproteins with pTyr leading to increased, decreased, normal, or abnormal signal transduction.

As used herein, the term “synergistic”, when applied to a combination of a stem and/or progenitor cell growth-promoting agent and a second agent, refers to a combination of a stem and/or progenitor cell growth-promoting agent and a second agent which is more effective than the additive effects of any two or more single agents. A synergistic effect of a combination of a stem and/or progenitor cell growth-promoting agent and one or more second agents permits the use of lower concentrations of the stem and/or progenitor cell growth-promoting agent and/or the one or more second agents. More effective relates to increased and improved, respectively, proliferation, self-renewal, and/or maintenance without differentiation of the stem and/or progenitor cells.

Abbreviation
cAMP Cyclic Adenosine Monophosphate
CNS Central Nervous System
GPCR G-protein coupled receptor
MAO Monoamine Oxidase
COMT Catechol-O-Methyltransferase
IP3 inositol-1,4,5-triphosphate
Kᵢ Inhibitor constant

### 5. DETAILED DESCRIPTION OF THE INVENTION

The present invention provides improved methods for culturing and maintaining stem cells. In certain, more specific embodiments, the invention provides methods for culturing fetal stem cells in the presence of a stem and/or progenitor cell growth-modulating agent. In certain, more specific embodiments, the invention provides methods for culturing adult stem cells in the presence of a stem and/or progenitor cell growth-modulating agent. In certain embodiments, the invention provides improved methods for culturing neural stem cells in the presence of a stem and/or progenitor cell growth-modulating agent. In certain, more specific embodiments, the invention provides methods for culturing fetal neural stem cells in the presence of a stem and/or progenitor cell growth-modulating agent. In certain, more specific embodiments, the invention provides methods for culturing adult neural stem cells in the presence of a stem and/or progenitor cell growth-modulating agent. In certain, more specific embodiments, the invention provides methods for culturing embryonal stem cells in the presence of a stem and/or progenitor cell growth-modulating agent. In certain, the invention provides methods for culturing progenitor cells in the presence of a stem and/or progenitor cell growth-modulating agent for different tissues. In certain, more specific embodiments, the invention provides methods for culturing fetal progenitor cells in the presence of a stem and/or progenitor cell growth-modulating agent. In certain, more specific embodiments, the invention provides methods for culturing adult progenitor cells in the presence of a stem and/or progenitor cell growth-modulating agent. In certain, more specific embodiments, the invention provides methods for culturing neural progenitor cells in the presence of a stem and/or progenitor cell growth-modulating agent. In certain, more specific embodiments, the invention provides methods for culturing fetal neural progenitor cells in the presence of a stem and/or progenitor cell growth-modulating agent. In certain, more specific embodiments, the invention provides methods for culturing adult neural progenitor cells in the presence of a stem and/or progenitor cell growth-modulating agent.

Further, the present invention provides improved methods for propagating stem cells. In certain, more specific embodiments, the invention provides methods for propagating fetal stem cells in the presence of a stem and/or progenitor cell growth-modulating agent. In certain, more specific embodiments, the invention provides methods for propagating adult stem cells in the presence of a stem and/or progenitor cell growth-modulating agent. In certain embodiments, the invention provides improved methods for propagating neural stem cells in the presence of a stem and/or progenitor cell growth-modulating agent. In certain, more specific embodiments, the invention provides methods for propagating fetal neural stem cells in the presence of a stem and/or progenitor cell growth-modulating agent. In certain, more specific embodiments, the invention provides methods for propagating adult neural stem cells in the presence of a stem and/or progenitor cell growth-modulating agent. In certain, more specific embodiments, the invention provides methods for propagating embryonal stem cells in the presence of a stem and/or progenitor cell growth-modulating agent. In certain, the invention provides methods for propagating progenitor cells in the presence of a stem and/or progenitor cell growth-modulating agent for different tissues. In certain, more specific embodiments, the invention provides methods for propagating fetal progenitor cells in the presence of a stem and/or progenitor cell growth-modulating agent. In certain, more specific embodiments, the invention provides methods for propagating adult progenitor cells in the presence of a stem and/or progenitor cell growth-modulating agent. In certain, more specific embodiments, the invention provides methods for propagating neural progenitor cells in the presence of a stem and/or progenitor cell growth-modulating agent. In certain, more specific embodiments, the invention provides methods for propagating fetal neural progenitor cells in the presence of a stem and/or progenitor cell growth-modulating agent. In certain, more specific embodiments, the invention provides methods for propagating adult neural progenitor cells in the presence of a stem and/or progenitor cell growth-modulating agent.

Further, the present invention provides improved methods for culturing, propagating, and/or generating neurospheres in vitro. In certain, more specific embodiments, the invention provides methods for generating neurospheres in vitro from neural stem cells. In certain, more specific embodiments, the invention provides methods for generating neurospheres in vitro from fetal neural stem cells. In certain, more specific embodiments, the invention provides methods for generating neurospheres in vitro from adult neural stem cells.

The invention provides methods for incubating stem cells in a defined tissue culture medium comprising a stem and/or progenitor cell growth-modulating agent. In certain embodiments, the invention provides methods for incubating adult and/or fetal stem cells in a defined tissue culture medium comprising a stem and/or progenitor cell growth-modulating agent. In certain embodiments, the invention provides methods for incubating adult and/or fetal stem cells in a defined tissue culture medium comprising a stem and/or progenitor cell growth-modulating agent. In certain embodiments, the invention provides methods for incubating adult and/or fetal neural stem cells in a defined tissue culture medium comprising a stem and/or progenitor cell growth-modulating agent. In certain embodiments, the invention provides methods for incubating adult and/or fetal progenitor cells in a defined tissue culture medium comprising a stem and/or progenitor cell growth-modulating agent. In certain embodiments, the invention provides methods for incubating adult and/or fetal neural progenitor cells in a defined tissue culture medium comprising a stem and/or progenitor cell growth-modulating agent.

In certain embodiments, the invention provides methods for culturing or maintaining stem cells or progenitor cells in the presence of a stem and/or progenitor cell growth-modulating agent and a second agent. Examples for such second agents are provided below in section 5.3.2.

In certain embodiments, the invention provides methods for amplifying stem cells and/or progenitor cells. In specific embodiments, the stem cells and or progenitor cells are amplified by culturing the stem cells and/or progenitor cells in the presence of a stem and/or progenitor cell growth-modulating agent.

In certain embodiments, the invention further provides methods for treating a disease or disorder of the nervous system, such as, inter alia, schizophrenia and epilepsia. In more specific embodiments, the invention provides methods for treating and/or preventing a neurodegenerative disease or disorder, such as, inter alia, Parkinson's disease and Alzheimer's disease. In certain embodiments, the methods of the invention for treating and/or preventing a neurodegenerative disorder comprise administering neural stem cells and/or neural progenitor cells that have been cultured using the methods of the present invention to a subject in need of treatment and/or prevention. In certain other embodiments, the methods of the invention for treating and/or preventing a neurodegenerative disorder comprise administering to a subject a stem and/or progenitor cell growth-modulating agent. In even other embodiments, the methods for treating and/or preventing a neurodegenerative disorder comprise administering a stem and/or progenitor cell growth-modulating agent and a neural stem cell that, preferably, has been cultured using methods of the present invention. In certain specific embodiments, the methods for treating and/or preventing a neurodegenerative disorder comprise administering a stem and/or progenitor cell growth-modulating agent that inhibits PTPases involved in the regulation receptor tyrosine kinase signaling pathway in neural stem cells or neural progenitor cells. In certain specific embodiments, the methods for treating and/or preventing a neurodegenerative disorder comprise administering a stem and/or progenitor cell growth-modulating agent that inhibits phosphate binding enzymes, binding proteins and/or receptor signaling pathways in neural stem cells or neural progenitor cells.

Modulation exerted by certain of the compounds that are to be used with the methods of the invention may in part be attributed to modulation of enzyme activities not displaying PTPase characteristics. Such enzymes can be, but are not limited to, phosphate binders like: glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases.

In certain embodiments, the invention provides methods for treating and/or preventing a disease or disorder involving neural stem cells. Such diseases and/or disorder include, but are not limited to, Parkinson's Disease, Alzheimer's Disease, Amyotrophic Lateral Sclerosis, Multiple Sclerosis, Spinal Cord Injury (as caused by, e.g., infection, inflammation, trauma, cancer, osteoporosis), Stroke, Depression, Drug abuse, diseases and/or disorder affecting memory. In specific embodiments, the methods of the invention include treating and/or preventing ageing deficits and obesity. In certain embodiments, the invention provides methods for regulating eating behaviour.

In certain embodiments, the methods of the invention for treating and/or preventing a disease or disorder involving neural stem cells comprise administering neural stem cells and/or neural progenitor cells that have been cultured using the methods of the present invention to a subject in need of treatment and/or prevention. In certain other embodiments, the methods of the invention for treating and/or preventing a disease or disorder involving neural stem cells comprise administering to a subject a stem and/or progenitor cell growth-modulating agent. In even other embodiments, the methods for treating and/or preventing a disease or disorder involving neural stem cells comprise administering a stem and/or progenitor cell growth-modulating agent and a neural stem cell that, preferably, has been cultured using methods of the present invention. In certain specific embodiments, the methods for treating and/or preventing a disease or disorder involving neural stem cells comprise administering a stem and/or progenitor cell growth-modulating agent that inhibits PTPases involved in the regulation receptor tyrosine kinase signaling pathway in neural stem cells or neural progenitor cells. In certain specific embodiments, the methods for treating and/or preventing a disease or disorder involving neural stem cells comprise administering a stem and/or progenitor cell growth-modulating agent that inhibits phosphate binding enzymes, binding proteins and/or receptor signaling pathways in neural stem cells or neural progenitor cells.

In certain embodiments, the invention provides methods for treating and/or preventing a disease or disorder involving stem cells and/or progenitor cells. Such diseases and/or disorder include, but are not limited to, Parkinson's Disease, Alzheimer's Disease, Amyotrophic Lateral Sclerosis, Multiple Sclerosis, Spinal Cord Injury (as caused by, e.g., infection, inflammation, trauma, cancer, osteoporosis), Stroke, Depression, Drug abuse, diseases and/or disorder affecting memory. In specific embodiments, the methods of the invention include treating and/or preventing ageing deficits and obesity. In certain embodiments, the invention provides methods for regulating eating behaviour. In certain embodiments, the methods of the invention for treating and/or preventing a disease or disorder involving stem cells and/or progenitor cells comprise administering stem cells and/or progenitor cells of the type that is affected in the disease or disorder that is to be treated, where the stem cells and/or progenitor cells that are to be administered have been cultured using the methods of the present invention. In certain other embodiments, the methods of the invention for treating and/or preventing a disease or disorder involving stem cells and/or progenitor cells comprise administering to a subject a stem and/or progenitor cell growth-modulating agent. In even other embodiments, the methods for treating and/or preventing a disease or disorder involving neural stem cells comprise administering a stem and/or progenitor cell growth-modulating agent and a stem cell and/or progenitor cell, where the stem cell and/or progenitor cell is of the same type as the stem cell and/or progenitor cell that is affected in the disease or disorder that is to be treated and/or prevented, and where the stem cell and/or progenitor cell, preferably, has been cultured using methods of the present invention. In certain specific embodiments, the methods for treating and/or preventing a disease or disorder involving stem cells and/or progenitor cells comprise administering a stem and/or progenitor cell growth-modulating agent that inhibits PTPases involved in the regulation of a receptor tyrosine kinase signaling pathway in neural stem cells or neural progenitor cells. In certain specific embodiments, the methods for treating and/or preventing a disease or disorder involving stem cells and/or progenitor cells comprise administering a stem and/or progenitor cell growth-modulating agent that inhibits phosphate binding enzymes, binding proteins and/or receptor signaling pathways in neural stem cells or neural progenitor cells.

As used in the context of the present invention, stem cells include, but are not limited to, stem cells of endothelial, mesenchymal, epithelial, haemopoietic, pancreatic, and muscular origins. Stem cells of endoderm origin include, but are not limited to, stem cells of gut, pancreas, and liver. Stem cells of ectoderm origin include, but are not limited to, stem cells of epidermal tissue and the nervous system. Stem cells of Mesoderm origin include, but are not limited to, muscle, bone, and blood.

In certain embodiments, the invention provides methods for culturing and maintaining a stem cell such that the stem cell does not differentiate. In more specific embodiments, the invention provides methods for culturing and maintaining an adult stem cell such that the adult stem cell does not differentiate. In more specific embodiments, the invention provides methods for culturing and maintaining an embryonal stem cell such that the embryonal stem cell does not differentiate. In certain embodiments, the invention provides methods for culturing and maintaining a neural stem cell such that the neural stem cell does not differentiate. In more specific embodiments, the invention provides methods for culturing and maintaining an adult neural stem cell such that the adult neural stem cell does not differentiate. In more specific embodiments, the invention provides methods for culturing and maintaining an embryonal neural stem cell such that the embryonal neural stem cell does not differentiate.

In certain specific embodiments, stem cells or progenitor cells are treated, cultured or maintained in minimal defined growth medium containing a stem and/or progenitor cell growth-modulating agent. In a more specific embodiment, stem cells or progenitor cells are treated, cultured or maintained in minimal defined growth medium containing a phosphate vanadate.

In certain embodiments, stem cells or progenitor cells are treated, propagated, cultured or maintained in tissue culture medium that has not been supplemented with exogenously added protein growth factors.

In certain embodiments, stem cells and/or progenitor cells are treated, propagated, cultured or maintained in tissue culture medium that has not been supplemented with exogenously added FGF, EGF, PDGF, NGF, IGF-1 and other IGF variants, Growth Hormone, FGF-acidic, FGF-basic, FGF-5, FGF-8b, FGF-17, FGF-18 and other FGF variants, VEGF165 and other VEGFs, PDGF-AA, PDGF-BB and other variants of PDGF (homodimers CC, DD etc and heterodimers AB, AC etc), BMP-2, BMP-4, BMP 6, BMP7 and other BMP variants, TGF-beta1, TGFbeta2, TGF-beta3, Activin A, TGFa, EGF, Amphiregulin, GDNF, BDNF, CNTF, Sonic hedgehog, NT-4, NT-3, b-NGF, CSFs (colony stimulating factors), Erythropoietin, TNFalpha, IL-1 alpha, IL-1beta, IL-6, IL-11, RANK Ligand/TRANCE/TNFSF11, Interferon-alpha-a, Interferon-gamma and other IFN variants, LIF, Neurturin, and Cell-bound “factors”/ligands, such as, but not limited to, Ephrin-A3, Ephrin-A5, Ephrin A7, Ephrin-B2 and other Ephrin variants.

In certain specific embodiments, the stem cells or progenitor cells are human stem cells or human progenitor cells. In a specific embodiment of the invention, human neural stem cells are used.

In certain embodiments, the invention provides a therapeutic method for inhibiting PTPases involved in the regulation receptor tyrosine kinase signaling pathway in neural stem cells or neural progenitor cells in human or animal stem cell derived diseseases and dysfunctions.

In certain embodiments, the invention provides a therapeutic method for inhibiting PTPases involved in the regulation of a receptor tyrosine kinase signaling pathway in stem cells or progenitor cells in human or animal stem cell derived diseases and dysfunctions.

In certain embodiments, the invention provides a therapeutic method for inhibiting phosphate binding enzymes, binding proteins and receptor signaling pathways in neural stem cells or neural progenitor cells in human or animal stem cell derived diseases and dysfunctions.

In certain embodiments, the invention provides a therapeutic method for inhibiting phosphate binding enzymes, binding proteins and receptor signaling pathways in stem cells or progenitor cells in human or animal stem cell derived diseases and dysfunctions.

In certain embodiments, the invention provides a method for proliferating and maintaining neural stem cell and neural progenitor cultures for the purpose of using the cells in therapy, drug discovery or diagnostics.

In certain embodiments, the invention provides a method for proliferating and maintaining stem cell and progenitor cultures for the purpose of using the cells in therapy, drug discovery or diagnostics.

5.1 Long-Term Maintenance and Amplification of Stem Cells without Differentiation

In certain embodiments, the invention provides methods for the maintenance of stem cells and/or progenitor cells. In certain embodiments, the invention provides methods for the long-term maintenance of stem cells and/or progenitor cells. In particular, the invention provides methods for maintaining, or culturing of stem cells and/or progenitor cells in the presence of a stem and/or progenitor cell growth-modulating agent for a certain period of time. The period of time is at least 1 day, 2 days, 3 days, 4 days, 5 days, 6 days, 1 week, 2 weeks, 3 weeks, 1 month, 2 months, 3 months, 6 months, 9 months, 1 year, 2 years, 5 years or 10 years. In certain embodiments, the period of time is at most 1 day, 2 days, 3 days, 4 days, 5 days, 6 days, 1 week, 2 weeks, 3 weeks, 1 month, 2 months, 3 months, 6 months, 9 months, 1 year, 2 years, 5 years or 10 years.

Several assays are available to the skilled artisan to test that the stem cells and/or progenitor cells are still stem cells and/or progenitor cells after the time period of maintaining or culturing. Examples of such assays are described below and include, inter alia, self-renewal assays and tests for differentiation. Other assays are well-known to the skilled artisan and can also be used with the methods of the invention.

In certain embodiments, the cells are treated with a stem and/or progenitor cell growth-modulating agent for a certain period of time and are maintained subsequently in the absence of the stem and/or progenitor cell growth-modulating agent. The certain period of time can be at least 1 hour, 2 hours, 5 hours, 12 hours, 1 day, 2 days, 3 days, 4 days, 5 days, 6 days, 1 week, 2 weeks, 3 weeks, 1 month, 2 months, 3 months, 6 months, 9 months, 1 year, 2 years, 5 years or 10 years. In certain embodiments, the certain period of time is at most 2 hours, 5 hours, 12 hours, 1 day, 2 days, 3 days, 4 days, 5 days, 6 days, 1 week, 2 weeks, 3 weeks, 1 month, 2 months, 3 months, 6 months, 9 months, 1 year, 2 years, 5 years or 10 years.

In certain embodiments, the methods of the invention provide improved survival of a stem cell or a progenitor cell in tissue culture. In certain more specific embodiments, the methods of the invention improve cell survival in tissue culture by at least 5%, 10%, 20%, 30%, 40%, 50%, 75%, 100%, or at least 250%. In certain more specific embodiments, the methods of the invention improve cell survival in tissue culture by at most 5%, 10%, 20%, 30%, 40%, 50%, 75%, 100%, or at least 250%.

In certain embodiments, the methods comprise self-renewal of the stem cells and/or progenitor cells by culturing the stem cells and/or progenitor cells in the presence of a stem and/or progenitor cell growth-modulating agent. In certain embodiments of the invention, culturing stem cells and/or progenitor cells in the presence of a stem and/or progenitor cell growth-modulating agent results in at least 5%, 10%, 20%, 30%, 40%, 50%, 75%, 100%, or at least 250% more stem cells and/or progenitor cells to self-renew compared to culturing the stem cells and/or progenitor cells under otherwise the same conditions without the stem and/or progenitor cell growth-modulating agent. In certain embodiments of the invention, culturing stem cells and/or progenitor cells in the presence of a stem and/or progenitor cell growth-modulating agent results in at most 5%, 10%, 20%, 30%, 40%, 50%, 75%, 100%, or at most 250% more stem cells and/or progenitor cells to self-renew compared to culturing the stem cells and/or progenitor cells under otherwise the same conditions without the stem and/or progenitor cell growth-modulating agent.

In certain embodiments, ATP levels are measured to determine the number of cells in a certain volume of cell culture. In certain, more specific embodiments, ATP levels are measured to determine the number of viable cells per volume of cell culture. In certain embodiments, the invention provides a method for culturing a population of stem cells or progenitor cells, respectively, said method comprising incubating the stem cells or progenitor cells, respectively, in tissue culture medium comprising a phosphate mimic or a stem and/or progenitor cell growth-modulating agent, wherein the incubating step elevates ATP levels per a certain volume of culture, e.g., per 1 ml of culture, by at least 25%, 50%, 100%, 200%, 300% or at least 400% compared to ATP levels per the certain volume of cell culture, e.g. per ml of culture, without phosphate mimic or a stem and/or progenitor cell growth-modulating agent under otherwise same conditions. In certain embodiments, the invention further provides a method for culturing a population of stem cells or progenitor cells, respectively, said method comprising incubating the stem cells or progenitor cells, respectively, in tissue culture medium comprising a phosphate mimic or a stem and/or progenitor cell growth-modulating agent, wherein the incubating step elevates the total ATP level of the plurality of cells per a certain volume of culture, e.g., per 1 ml of culture, by at least 25%, 50%, 100%, 200%, 300% or at least 400% compared to the total ATP level of the plurality of cells per the certain volume of cell culture without phosphate mimic or a stem and/or progenitor cell growth-modulating agent under otherwise same conditions. The certain volume of culture can be any volume or any unit of volume, e.g., 1 nanoliter, 1 microliter, 1 milliliter, 1 liter etc.

5.1.1 The Tissue Culture Medium

In certain embodiments of the invention, the tissue culture medium is growth medium supplemented with BIT (growth cocktail containing BSA, insulin, and transferring Stem Technologies, Vancouver, Canada).

In certain embodiments, the tissue culture medium comprises at least 0.1 μg/ml, 1 μg/ml, 10 μg/ml, 100 μg/ml, 1 mg/ml or at least 10 mg/ml insulin. In certain embodiments, the tissue culture medium contains at most 0.1 μg/ml, 1 μg/ml, 10 μg/ml, 100 μg/ml, 1 mg/ml or at most 10 mg/ml insulin.

In certain embodiments, the tissue culture medium comprises at least 0.1 μg/ml, 1 μg/ml, 10 μg/ml, 100 μg/ml, 1 mg/ml, or at least 10 mg/ml BSA. In certain embodiments, the tissue culture medium contains at most 0.1 μg/ml, 1 μg/ml, 10 μg/ml, 100 μg/ml, 1 mg/ml or at most 10 mg/ml BSA.

In certain embodiments, the tissue culture medium comprises at least 0.1 μg/ml, 1 μg/ml, 10 μg/ml, 100 μg/ml, or at least 1 mg/ml transfernin. In certain embodiments, the tissue culture medium contains at most 0.1 μg/ml, 1 μg/ml, 10 μg/ml, 100 μg/ml, or at most 1 mg/ml transferrin.

In certain embodiments, the tissue culture medium is supplemented with B27 (Gibco BRL, Invitrogen).

In certain embodiments, the tissue culture medium is not supplemented with any exogenously added growth factors.

In certain embodiments, the growth factors are used at a concentration that, in the absence of a stem and/or progenitor cell growth-modulating agent, does not support the proliferation, or the propagation, or the renewal of the stem cell or progenitor cell.

In certain embodiments, the tissue culture medium does not comprise insulin. In certain embodiments, the tissue culture medium does not comprise BSA. In certain embodiments, the tissue culture medium does not comprise transferrin.

In certain embodiments, the invention provides methods for the proliferation, culturing, or maintaining of stem cells and/or progenitor cells with reduced amounts of growth factors, such as EGF, FGF-2, FGF-8, VEGF, IGF, NGF, heparin, erythropoietin, interleukins, interferons, and LIF. In certain embodiments, the concentration of EGF, FGF-2, FGF-8, VEGF, IGF, NGF, heparin, erythropoietin, interleukins, interferons, or LIF is at most 0.1 ng/ml, 1 ng/ml, 10 ng/ml, 0.1 μg/ml, 1 μg/ml, 10 μg/ml, 100 μg/ml. In certain embodiments, the invention provides methods for the proliferation, culturing, maintaining of stem cells and/or progenitor cells with reduced amounts of insulin. In certain embodiments, the concentration of insulin is at most 0.1 ng/ml, 1 ng/ml, 10 ng/ml, 0.1 μg/ml, 1 μg/ml, 10 μg/ml, 100 μg/ml.

In certain embodiments, the stem cells and/or progenitor cells are proliferated, cultured, and/or maintained in medium that has been supplemented with at least 1%, 2%, 3%, 4%, 5%, 6%, 8%, 9%, 10%, 12%, 15%, or at least 20% of serum. In certain embodiments, the stem cells and/or progenitor cells are proliferated, cultured, and/or maintained in medium that has been supplemented with at most 1%, 2%, 3%, 4%, 5%, 6%, 8%, 9%, 10%, 12%, 15%, or at most 20% of serum. In specific embodiments, the serum is, e.g., fetal calf serum. In specific embodiments, the serum is heat-inactivated.

In certain embodiments, the stem cells and/or progenitor cells are proliferated, cultured, and/or maintained in medium that has not been supplemented with exogenously added serum.

In certain embodiments, the medium is defined medium. In certain embodiments, the medium is not defined medium.

5.2 Stimulated Differentiation

In certain embodiments, the invention provides methods for the stimulated differentiation of stem cells and/or progenitor cells.

In certain embodiments, the cells are treated, maintained, or cultured in the presence of a stem and/or progenitor cell growth-modulating agent and a differentiation stimulating agent. In certain embodiments, the differentiation stimulating agent triggers the stem cells and/or progenitor cells to differentiate to, e.g., neural, hematopoietic, cardilage, bone, or epithelial tissue/cells. In certain specific embodiments, the differentiating agents include, but are not limited to, PDGF AA, BB, AB, BDNF, CNTF GDNF, NT3, NT4, sonic hedge-hog, FGF-8, retinoic acid, forskolin.

In certain other embodiments, the cells are treated, maintained, or cultured in the presence of a stem and/or progenitor cell growth-modulating agent and are subsequently treated, cultured or maintained with a differentiation stimulating agent in the absence of a stem and/or progenitor cell growth-modulating agent.

5.3. Stem and/or Progenitor Cell Growth-Modulating Agents

A stem and/or progenitor cell growth-modulating agent is a substance selected from the group consisting of inhibitors of PTPases, modulators of enzymes with phosphate binding sites, modulators of activities of proteins with pTyr recognition units, and phosphate mimics. Enzymes with phosphate binding sites are, e.g., glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases. pTyr recognition units include, but are not limited to, SH2 domains. In a preferred embodiment, Enzymes with phosphate binding sites are selected from the group consisting of glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases. In a specific embodiment, a pTyr recognition unit is a SH2 domain.

Examples of modulators of enzymes with phosphate binding sites are: rolipram as modulator of phosphodiesterase IV; sildenafil as modulator of phosphodiesterase V; ouabain as modulator of Na/K ATPase; aurintricarboxylic acid as modulator of phosphofructokinase; staurosporin as modulator of protein kinase C; genistein as modulator of protein-Tyr kinase; gleevec(imatinib) as modulator of protein-Tyr kinase; di(t-butyl)-1,4-quinone as modulator of Ca-ATPase; and thapsigargin as modulators of Ca-ATPase.

Peptides preventing the binding reaction between the p-Tyr recognition unit and its binding partner can be used as modulators of proteins with p-Tyr recognition units.

In certain embodiments, the agent to be used with the methods of the invention, i.e., the stem and/or progenitor cell growth-modulating agent, is an inhibitor of PTPases, e.g., protein tyrosine phosphatases involved in the regulation of tyrosine kinase signaling pathways. PTPases include, but are not limited to, PTP1B, CD45, SHP-1, SHP-2, PTPalpha, LAR and HePTP. Examples of PTPase inhibiting compounds are disclosed in U.S. Pat. Nos. 6,388,076; 6,262,044; 6,225,329; 6,169,087; 6,040,323; 5,925,660; 5,877,210; 5,798,374; 5,693,627, which are incorporated by reference in their entireties herein for all purposes, including the description of the methods of making, using, and formulating PTPase inhibiting compounds. In certain embodiments the agent to be used with the methods of the invention modulates receptor-tyrosine kinase signaling pathways via interaction with regulatory PTPases, e.g., the signaling pathways of the insulin receptor, the IGF-I receptor and other members of the insulin receptor family, the EGF-receptor family, the platelet-derived growth factor family, the nerve growth factor receptor family, the hepatocyte growth factor receptor family, the growth hormone receptor family and members of other receptor-type tyrosine kinase families. In certain embodiments, the agent to be used with the methods of the invention modulates non-receptor tyrosine kinase signaling through modulation of regulatory PTPases, e.g., modulation of members of the Src kinase family. In more specific embodiments, the agent to be used with the methods of the invention modulates the activity of PTPases that negatively regulate signal transduction pathways. In other embodiments, the agent to be used with the methods of the invention modulates the activity of PTPases that positively regulate signal transduction pathways.

In certain embodiments, the agent to be used with the methods of the invention modulates enzyme activities not displaying PTPase characteristics. Such enzymes can be, but are not limited to, phosphate binders like: glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases.

In certain embodiments, the agent to be used with the methods of the invention modulates the activity of PTPases via interaction with the active site of PTPases. In certain embodiments, the agent to be used with the methods of the invention modulates the activity of PTPases via interaction with structures positioned outside the active sites of the enzymes, e.g., SH2 domains. In certain embodiments, the agent to be used with the methods of the invention modulates signal transduction pathways via binding of the compounds of the invention to SH2 domains or PTB domains of non-PTPase signaling molecules.

In certain embodiments, the agent to be used with the methods of the invention modulates cell-cell interactions as well as cell-matrix interactions.

In certain embodiments, the agent to be used with the methods of the invention is a phosphate mimic (see section 5.3.1).

The optimum concentration of the agent to be used with the methods of the invention in the culture of stem cells can easily be determined by culturing the stem cells under otherwise the same conditions in the presence of different concentrations of the phosphate mimic. The optimum concentration will vary with the nature of the agent to be used with the methods of the invention and the desired effect, i.e., whether minimal differentiation of the cells, maximal proliferation, self renewal, or formation of neurospheres is desired. The same conditions relate inter alia to the following parameters: approximately the same cell density at the beginning of the assay; the same temperature, culture medium, CO₂ concentration, same period of time of the different incubation and culturing steps.

5.3.1 Phosphate Mimics

In certain embodiments, the agent to be used with the methods of the invention is a phosphate mimic. A phosphate mimic is an agent that is structurally similar to a phosphate molecule such that is binds to sites to which a phosphate binds. Such phosphate binding sites include, but are not limited to, the phosphate binding sites of glucose-6-phosphate dehydrogenase, fructose-2,6-bisphosphatase, phosphoglucomutase, Mg dependent ATPase, plasma membrane Ca ATPases, endoplamic, reticulum Ca2+-ATPases, P-glycoprotein ATPase activity, Mg(2+)-dependent vanadate-sensitive GS-conjugate export ATPase, MRP/GS-X pump, Na+, K+-ATPase as well as of other related and unrelated phosphohydrolases, pyrophosphatase, alkaline and acid phosphatases. In certain embodiments, a phosphate mimic is a molecule that comprises a moiety that is structurally similar to a phosphate such that the phosphate mimic is capable of binding to a phosphate binding site.

The binding of a phosphate mimic to a phosphate binding site in a protein can be assayed by Nuclear Magnetic Resonance.

In certain embodiments, the phosphate mimic is a vanadium oxide, a derivative of a vanadium oxide, a polyoxometalate, a homopolyoxotungstate, a vanadium-substituted polyoxotungstate, an esterified derivative of 4-(fluoromethyl)phenyl phosphate, a homopolyoxoselenate, a vanadium-substituted polyoxoselenate, a homopolyoxomolybdate, a vanadium-substituted polyoxomolybdate, and a PTPase inhibitor.

In certain more specific embodiments, the phosphate mimic is vanadate, orthovanadate, metavanadate, pervanadate; vanadate dimer, vanadate tetramer, vanadate pentamer, vanadate hexamer, vanadate heptamer, vanadate octamer, vanadate nonamer, vanadate decamer, vanadate polymer, vanadyl sulfate, bis(6, ethylpicolinato)(H(2)O)oxovanadium(IV) complex, bis(1-oxy-2-pyridinethiolato)oxovanadium(IV), bis(maltolato)oxovanadium (IV), bis(biguanidato)oxovanadium(IV), bis(N′N′-dimethylbiguanidato)oxovanadium(IV), bis(beta-phenethyl-biguanidato)oxovanadium(IV), peroxovanadate-nicotinic acid, aluminiofluoride, 4-(fluoromethyl)phenyl phosphate, tungstate, selenate, molybdate, Zn²⁺ or F¹⁻.

In certain specific embodiments, the phosphate mimic is a PTPase inhibiting compound. Examples of PTPase inhibiting compounds are disclosed in U.S. Pat. Nos. 6,388,076; 6,262,044; 6,225,329; 6,169,087;6,040,323; 5,925,660; 5,877,210; 5,798,374; 5,693,627, which are incorporated by reference in their entireties herein for all purposes, including the description of the methods of making, using, and formulating PTPase inhibiting compounds.

In specific embodiments of the invention, vanadate is added to the cell culture as sodium ortho-vanadate.

The optimum concentration of the phosphate mimic in the culture of stem cells can easily be determined by culturing the stem cells under otherwise the same conditions in the presence of different concentrations of the phosphate mimic. The optimum concentration will vary with the nature of the phosphate mimic and the desired effect, i.e., whether minimal differentiation of the cells, maximal proliferation, self renewal, or formation of neurospheres is desired. The same conditions relate inter alia to the following parameters: approximately the same cell density at the begining of the assay; the same temperature, culture medium, CO₂ concentration, same period of time of the different incubation and culturing steps.

In a certain embodiments, the phosphate mimic is vanadate. In certain specific embodiments, the concentration of vanadate in the culture medium is at least 100 nM, at least 1 μM, at least 4 μM, at least 10 μM, at least 50 μM, at least 100 μM, at least 250 μM, at least 500 μM, at least 1 mM or at least 10 mM. In certain specific embodiments, the concentration of vanadate in the culture medium is at most 100 nM, at most 1 μM, at most 4 μM, at most 10 μM, at most 50 μM, at most 100 μM, at most 250 μM, at most 500 μM, at most 1 mM or at most 10 mM. In certain embodiments, the concentration of vanadate in the culture medium is between 100 nM and 1 μM, 1 μM and 10 μM, 10 μM and 50 μM, 50 μM and 100 μM, 100 μM and 250 μM, 250 μM and 500 μM, or between 500 μM and 1 mM.

In certain specific embodiments, the phosphate mimic is an inhibitor of PTPases, e.g., protein tyrosine phosphatases involved in the regulation of tyrosine kinase signaling pathways. PTPases include, but are not limited to, PTP1B, CD45, SHP-1, SHP-2, PTPalpha, LAR and HePTP. In certain specific embodiments the phosphate mimic modulates receptor-tyrosine kinase signaling pathways via interaction with regulatory PTPases, e.g., the signaling pathways of the insulin receptor, the IGF-I receptor and other members of the insulin receptor family, the EGF-receptor family, the platelet-derived growth factor family, the nerve growth factor receptor family, the hepatocyte growth factor receptor family, the growth hormone receptor family and members of other receptor-type tyrosine kinase families. In certain specific embodiments, the phosphate mimic modulates non-receptor tyrosine kinase signaling through modulation of regulatory PTPases, e.g., modulation of members of the Src kinase family. In more specific embodiments, the phosphate mimic modulates the activity of PTPases that negatively regulate signal transduction pathways. In other specific embodiments, the phosphate mimic modulates the activity of PTPases that positively regulate signal transduction pathways.

In certain specific embodiments, the phosphate mimic modulates the activity of PTPases via interaction with the active site of PTPases. In certain specific embodiments, the phosphate mimic modulates the activity of PTPases via interaction with structures positioned outside the active sites of the enzymes, e.g., SH2 domains. In certain specific embodiments, the phosphate mimic modulates signal transduction pathways via binding of the compounds of the invention to SH2 domains or PTB domains of non-PTPase signaling molecules.

In certain embodiments, the phosphate mimic modulates cell-cell interactions as well as cell-matrix interactions.

5.3.2 Agents to be Used in Combination with the Stem and/or Progenitor Cell Growth-Modulating Agents

The invention provides methods for incubating stem cells in a tissue culture medium comprising a stem and/or progenitor cell growth-modulating agent and a second agent. The invention provides methods for incubating stem cells in a defined tissue culture medium comprising a stem and/or progenitor cell growth-modulating agent and a second agent. In certain embodiments, the invention provides methods for incubating adult and/or fetal stem cells in a defined tissue culture medium comprising a stem and/or progenitor cell growth-modulating agent and a second agent. In certain embodiments, the invention provides methods for incubating adult and/or fetal stem cells in a defined tissue culture medium comprising a stem and/or progenitor cell growth-modulating agent and a second agent. In certain embodiments, the invention provides methods for incubating adult and/or fetal neural stem cells in a defined tissue culture medium comprising a stem and/or progenitor cell growth-modulating agent and a second agent. In certain embodiments, the invention provides methods for incubating adult and/or fetal progenitor cells in a defined tissue culture medium comprising a stem and/or progenitor cell growth-modulating agent and a second agent. In certain embodiments, the invention provides methods for incubating adult and/or fetal neural progenitor cells in a defined tissue culture medium comprising a stem and/or progenitor cell growth-modulating agent and a second agent.

A second agent can be, but is not limited to, a growth factor, a growth factor mimic acting on tyrosine kinase receptors, and growth factor secretagogues. A second agent further includes an agonist of cAMP accumulation. Agonists of cAMP accumulation include, inter alia, GPCR agonists and antagonists, i.e., agonists of activating G-protein coupled receptors and antagonists of inactivating G-protein coupled receptors, stimulators of adenylate cyclase and inhibitors of phosphodiesterase activity, neurotransmitter uptake blockers, MAO inhibitors, COMT inhibitors, neuropeptide peptidase inhibitors.

A second agent can further be a Ca-transient triggering factors. Ca-transient triggering factors includes, but are not limited to GPCR agonists and antagonists, Li-salts, sarcoplamic reticulum Ca-ATPase inhibitors, IP3 and IP3 receptor agonists, Ca ionophores, cell membrane depolarising agents, neurotransmitter uptake blockers, neuropeptide peptidase inhibitors, MAO inhibitors, and COMT inhibitors.

A second agent can also be an agonist of cGMP accumulation. Agonists of cGMP accumulation include, but are not limited to GPCR agonists and antagonists, stimulators of guanylate cyclase and inhibitors of phosphodiesterase activity, neurotransmitter uptake blockers, MAO inhibitors, COMT inhibitors, neuropeptide peptidase inhibitors; and natriuretic peptides and mimics thereof.

In certain embodiments, the stem and/or progenitor cell growth-modulating agent and the second agent act synergistically. In certain embodiments, the stem and/or progenitor cell growth-modulating agent and the second agent have synergistic effects on cell proliferation, cell self-renewal and/or cell survival.

5.4. Assays for Use with the Invention

In certain embodiments, the assays provided by the invention can be used for screening different substances for the effects on self-renewal of stem or progenitor cells, proliferation of stem or progenitor cells, and/or differentiation of stem or progenitor cells.

5.4.1 Cell Survival Assay

In certain embodiments, cell survival is determined after a period of time of incubating, culturing, maintaining, or propagating cells. In order to compare the effect of a stem and/or progenitor cell growth-modulating agent on cell survival, two populations of cells are incubated, cultured, maintained, or propagated, one in the presence and the other in the absence of a stem and/or progenitor cell growth-modulating agent, under otherwise the same conditions. The same conditions relate inter alia to the following parameters: approximately the same cell density at the beginning of the assay; the same temperature, culture medium, CO₂ concentration, same period of time of the different incubation and culturing steps. The time period is at least 4 hours, 8 hours, 12 hours, 18 hours, 24 hours, 2 days, 3 days, 4 days, 5 days, 6 days, 7 days, 2 weeks, 3 weeks, 4 weeks, 6 weeks, 2 months, 3 months, or at least 6 months. In certain embodiments, the time period is at most 4 hours, 8 hours, 12 hours, 18 hours, 24 hours, 2 days, 3 days, 4 days, 5 days, 6 days, 7 days, 2 weeks, 3 weeks, 4 weeks, 6 weeks, 2 months, 3 months, or at most 6 months. In a specific embodiment, cell survival is determined using alamarBlue™ (BioSource International, Inc.). Several hours before harvest, the cells are treated with alamarBlue™ (BioSource International, Inc.). By monitoring alamarBlue™ reduction spectrophotometrically, cell viability can be determined. Other techniques of determining cell viability are known to the skilled artisan and can be used with the methods of the present invention.

Improved cell survival relates to the increase in number of surviving cells in one population relative to the other population and is expressed in percent of that increase in one population compared to the number of surviving cells in the other population.

In certain embodiments, cell survival is determined by measuring intracellular ATP levels as described in sections 5.4.3 and 6.

5.4.2 Differentiation Assays

The differentiation state of a cell can be determined by any technique well-known to the skilled artisan. In general, the morphology of a cell and expression of specific marker genes by a cell can be used, inter alia, to determine the differentiation state of a cell. Morphological features include, but are not limited to, size of the cell, protrusions, attachment to a substrate, and the formation of aggregates. Morphological features can be scrutinized using, inter alia, light microscopy and electron microscopy.

The expression levels of marker genes in a cell can be tested by any method well-known to the skilled artisan. Such methods include, but are not limited to, Northern blot hybridization, Western blotting, in situ hybridization, immunohistochemistry, activity of cis-regulatory control elements in the cell.

Neuronal differentiation markers include, but are not limited to, tubulin β-III, NeuN, anti-tyrosine hydroxylase, anti-MAP-2 etc. As glial markers anti-GFAP, anti-S1100 etc. can be used. As oligodendrocyte markers anti-Ga1C, anti-PLP etc. can be used.

In certain embodiments, transmitter phenotypes can be measured by:

1) immunobased assays, histochemistry or ELISA/RIA/etc; transmitter concentration or enzyme (responsible for transmitter synthesis) concentration or activity (eg. Matute and Streit (1986) Histochemistry 86(2): 147-57); 2) chromatography-masspectrometry assays (or other detection principles eg electrochemical, fluorescence, absorbance) for concetration of transmitter (eg Sugita et al (2001) Int J Mol Med May; 7(5):521-5; 3) enzyme-based reactions generating chromophores, flourophores or luminescence for detection of transmitter concentration or enzyme activity (eg Haggblad et al (1983) J Neurochem. June; 40(6):1581-4; or 4) radiometry assays measuring radioactivity of enzyme products constituting transmitter or transmitter precursor or breakdown products (transmitter uptake and synthesis) (eg Ferrari et al (1991) J Neurosci Res November; 30(3):493-7).

In certain embodiments, the cis-acting control element of a gene that encodes a neural differentiation marker is cloned in front of a reporter gene. Reporter genes that can be used with the methods of invention include, but are not limited to, the genes listed in the Table 1 below:

TABLE 1
Reporter genes and the biochemical properties of the
respective reporter gene products

| Reporter Gene | Protein Activity & Measurement |
|---|---|
| CAT (chloramphenicol | Transfers radioactive acetyl groups |
| acetyltransferase) | to chloramphenicol |
| GAL (b-galactosidase) | Detection by thin layer chromato- |
| | graphy and autoradiography |
| GUS (b-glucuronidase) | Hydrolyzes colorless galactosides |
| | to yield colored products. |
| LUC (luciferase) | Hydrolyzes colorless glucuronides |
| | to yield colored products. |
| GFP (green fluorescent protein) | Oxidizes luciferin, emitting |
| | photons |
| SEAP (secreted alkaline phosphatase) | luminescence reaction with suitable |
| | substrates or with substrates that |
| | generate chromophores. |

In certain embodiments, the cis-acting control element-reporter gene DNA fragment is cloned into a vector that can be transfected into the cells that are to be analyzed. Transfection procedures are well-known to the skill artisan and include, but are not limited to, DEAE-dextran-mediated, Calcium phosphate-mediated, Electroporation, and Liposome-mediated transfection. The abundance and/or activity of the reporter gene is proportional to the activity of the cis-actin control element. The abundance of the reporter gene can be measured by, inter alia, Western blot analysis or Northern blot analysis or any other technique used for the quantification of transcription of a nucleotide sequence, the abundance of its mRNA its protein. In certain embodiments, the activity of the reporter gene product is measured as a readout of the transcriptional activity of the promoter sequence that is cloned in front of the nucleotide sequence encoding the reporter gene. For the quantification of the activity of the reporter gene product, biochemical characteristics of the reporter gene product can be employed (see Table 1). The methods for measuring the biochemical activity of the reporter gene products are well-known to the skilled artisan. Up-regulation of the reporter gene demonstrates that the promoter is more active and that the marker that is regulated by the same promoter is also up-regulated.

In certain other embodiments, the expression levels of an inhibitor of differentiation is assayed. Down-regulation of the inhibitor of differentiation demonstrates the ability of the cell to differentiate.

5.4.3 Proliferation Assays

Bromodeoxyuridine (BRDU) incorporation may, e.g. be used as an assay to identify and quantify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38, 369; Campana et al., 1988, J. Immunol. Meth. 107, 79). The percentage of cells with positive BRDU-staining is a parameter for the proliferative activity in a population of cells. To compare the proliferative activity of a cell population cultured in the presence of a stem and/or progenitor cell growth-modulating agent with the proliferative activity of a cell population that has been cultured in the absence of a stem and/or progenitor cell growth-modulating agent, the two cell populations are grown under otherwise the same conditions and BRDU incorporation is measured after a defined amount of time and the numbers of cells that are BRDU. The same conditions relate inter alia to the following parameters: approximately the same cell density at the beginning of the assay; the same temperature, culture medium, CO₂ concentration, same period of time of the different incubation and culturing steps.

Cell proliferation may also be examined using (³H)-thymidine incorporation (see e.g., Chen, J., 1996, Oncogene 13:1395-403; Jeoung, J., 1995, J. Biol. Chem. 270:18367-73). This assay allows for quantitative characterization of S-phase DNA synthesis. In this assay, cells synthesizing DNA will incorporate (³H)-thymidine into newly synthesized DNA. Incorporation may then be measured by standard techniques in the art such as by counting of radioisotope in a Scintillation counter (e.g., Beckman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189-199; Vassilev et al., 1995, J. Cell Sci. 108:1205-15).

Cell proliferation may be measured by counting samples of a cell population over time (e.g., daily cell counts). Cells may be counted using a hemacytometer and light microscopy (e.g., HyLite hemacytometer, Hausser Scientific). Cell number may be plotted against time in order to obtain a growth curve for the population of interest. In a preferred embodiment, cells counted by this method are first mixed with the dye Trypan Blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g., cells in S-phase) will exhibit a ploidy value higher than 2N and up to 4N DNA content. Ploidy value and cell-cycle kinetics may be further measured using propidum iodide assay (see, e.g., Turner, T., et al., 1998, Prostate 34:175-81). Alternatively, the DNA ploidy may be determined by quantitation of DNA Feulgen staining (which binds to DNA in a stoichiometric manner) on a computerized microdensitometrystaining system (see, e.g., Bacus, S., 1989, Am. J. Pathol. 135:783-92). In an another embodiment, DNA content may be analyzed by preparation of a chromosomal spread (Zabalou, S., 1994, Hereditas. 120:127-40; Pardue, 1994, Meth. Cell Biol. 44:333-351).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway may be indicated by the induction of p21^{cip1}. Increased levels of p21 expression in cells results in delayed entry into G1 of the cell cycle (Harper et al., 1993, Cell 75:805-816; Li et al., 1996, Curr. Biol. 6:189-199). p21 induction may be identified by immunostaining using a specific anti-p21 antibody available commercially (e.g., Santa Cruz). Similarly, cell-cycle proteins may be examined by Western blot analysis using commercially available antibodies. In another embodiment, cell populations are synchronized prior to detection of a cell cycle protein. Cell cycle proteins may also be detected by FACS (fluorescence-activated cell sorter) analysis using antibodies against the protein of interest.

Detection of changes in length of the cell cycle or speed of cell cycle may also be used to measure the effect of a stem and/or progenitor cell growth-modulating agent on the proliferation of stem cells and/or progenitor cells. In one embodiment the length of the cell cycle is determined by the doubling time of a population of cells (e.g., using cells that were cultured or maintained in the presence or in the absence of a stem and/or progenitor cell growth-modulating agent). In another embodiment, FACS analysis is used to analyze the phase of cell cycle progression, or purify G1, S, and G2/M fractions (see, e.g., Delia, D. et al., 1997, Oncogene 14:2137-47).

Lapse of cell cycle checkpoint(s), and/or induction of cell cycle checkpoint(s), may be examined by the methods described herein, or by any method known in the art. Without limitation, a cell cycle checkpoint is a mechanism which ensures that a certain cellular events occur in a particular order. Checkpoint genes are defined by mutations that allow late events to occur without prior completion of an early event (Weinert, T., and Hartwell, L., 1993, Genetics, 134:63-80). Induction or inhibition of cell cycle checkpoint genes may be assayed, for example, by Western blot analysis, or by immunostaining, etc. Lapse of cell cycle checkpoints may be further assessed by the progression of a cell through the checkpoint without prior occurrence of specific events (e.g., progression into mitosis without complete replication of the genomic DNA).

In addition to the effects of expression of a particular cell cycle protein, activity and post-translational modifications of proteins involved in the cell cycle can play an integral role in the regulation and proliferative state of a cell. Thus, assays for detecting post-translational modifications (e.g., phosphorylation and glycosylation) by any method known in the art can be used to determine the proliferative state of a cell population. For example, antibodies that detect phosphorylated tyrosine residues are commercially available, and may be used in Western blot analysis to detect proteins with such modifications. In another example, modifications such as myristylation, may be detected on thin layer chromatography or reverse phase H.P.L.C. (see, e.g., Glover, C., 1988, Biochem. J. 250:485-91; Paige, L., 1988, Biochem J.; 250:485-91).

Activity of signaling and cell cycle proteins and/or protein complexes is often mediated by a kinase activity. Thus, analysis of kinase activity by assays such as the histone H1 assay can also be used with the methods of the invention (see, e.g., Delia, D. et al., 1997, Oncogene 14:2137-47).

In certain embodiments, proliferation is quantified by (a) culturing one or more stem cell and/or progenitor cell populations in the presence of one or more concentrations of the stem and/or progenitor cell growth-modulating agent for a 72- to 96-hour period; (b) culturing one or more stem cell and/or progenitor cell populations without the stem and/or progenitor cell growth-modulating agent to the culture for a 72- to 96-hour period; (c) determining the number of viable stem cells and/or progenitor cells at the end of the 72- to 96-hour period in the stem cell and/or progenitor cell populations of step (a) and (b), respectively, and wherein the culturing steps (a) and (b) are conducted under otherwise the same conditions; and (d) determining the percent increase in the number of viable stem cells and/or progenitor cells of step (a) as compared to the number of viable stem cells of step (b).

Intracellular ATP levels can be measured to determine cell number. Intracellular ATP levels have previously been shown to correlate to cell number (Crouch, Kozlowski et al. 1993). In a specific embodiments, intracelluar ATP is measured using the ViaLight kit (BioWhittaker) according to the manufacturer's instructions after 3 days of incubation.

5.4.4 Assays for Secreted Factors

In certain embodiments, stem cells and/or progenitor cells that were treated, cultured or maintained in the presence of a stem and/or progenitor cell growth-modulating agent are tested for secretion of factors. In certain more specific embodiments, the stem cells and/or progenitor cells are treated, maintained or cultured in a medium comprising a stem and/or progenitor cell growth-modulating agent, subsequently the cells are spun down in a centrifuge and the supernatant is assayed for the presence of any secreted factors. In specific embodiments, the supernatant is assayed for the presence of secreted factors by, inter alia, Western blot analysis which is optionally preceded by immunoprecipitation. The Western blot analysis and the immunoprecipitation is conducted with antibodies specific to candidate secreted factors. Candidate secreted factors include, but are not limited to, EGF, FGF, NGF, PDGF, FGF, EGF, PDGF, NGF, IGF-1 and other IGF variants, Growth Hormone, FGF-acidic, FGF-basic, FGF-5, FGF-8b, FGF-17, FGF-18 and other FGF variants, VEGF165 and other VEGFs, PDGF-AA, PDGF-BB and other variants of PDGF (homodimers CC, DD etc and heterodimers AB, AC etc), BMP-2, BMP-4, BMP 6, BMP7 and other BMP variants, TGF-beta1, TGFbeta2, TGF-beta3, Activin A, TGFa, EGF, Amphiregulin, GDNF, BDNF, CNTF, Sonic hedgehog, NT-4, NT-3, b-NGF, CSFs (colony stimulating factors), Erythropoietin, TNFalpha, IL-1 alpha, IL-1 beta, IL-6, IL-11, RANK Ligand/TRANCE/TNFSF11, Interferon-alpha-a, Interferon-gamma and other IFN variants, LIF, Neurturin, and Cell-bound “factors”/ligands, such as, but not limited to, Ephrin-A3, Ephrin-A5, Ephrin A7, Ephrin-B2 and other Ephrin variants.

5.4.5 Self-Renewal Assay and Colony-Forming Assay

In certain embodiments, the cells were assayed for self-renewal by dissociating the cells and reculturing the cells at a constant density of 20 cells per microliter. The assay can be performed as described in Tropepe et al, 1999, Dev Biol 208:166-188 or in Seaberg and van der Kooy, 2002, J Neurosci March 1; 22(5):1784-93.

The for colony forming assay can be performed as follows: Dissociated cells are seeded at constant number and evaluation of colony formation is based on the frequency of precursors that initiate neurosphere cultures. Assay can be performed as described in Reynolds and Weiss (1992) Science; 255(5052):1707-1710, Chiasson et al (1999) J Neurosci; 19(11):4462-4471, Uchida et al (2000) PNAS; 97(26):14720-14725.

5.4.6 In Vivo Assays

In certain embodiments of the invention, a stem and/or progenitor cell growth-modulating agent is administered to an animal to assay the effects of the stem and/or progenitor cell growth-modulating agent in vivo. In certain embodiments, the stem and/or progenitor cell growth-modulating agent is a to an animal model of a CNS disease, disorder, or trauma. Such CNS diseases, disorders, or traumas include, but are not limited to:

Models of epilepsia, such as: Electroshock-induced seizures (Billington A et al., Neuroreport 2000 Nov. 27; 11(17):3817-22), pentylene tetrazol (Gamaniel K et al., Prostaglandins Leukot Essent Fatty Acids 1989 February; 35(2):63-8) or kainic acid (Riban V et al, Neuroscience 2002; 112(1):101-11) induced seizures.

Models of psychosis/schizophrenia, such as: amphetamine-induced stereotypies/locomotion (Borison R L & Diamond B I, Biol Psychiatry 1978 April; 13(2):217-25), MK-801 induced stereotypies (Tiedtke et al., J Neural Transm Gen Sect 1990; 81(3):173-82), MAM (methyl azoxy methanol-induced (Fiore M et al., Neuropharmacology 1999 June; 38(6):857-69; Talamini L M et al., Brain Res 1999 Nov. 13; 847(1):105-20) or reeler model (Ballmaier M et al., Eur J Neurosci 2002 April; 15(17):1197-205).

Models of Parkinson's disease, such as: MPTP (Schmidt & Ferger, J Neural Transm 2001; 108(11):1263-82), 6-OH dopamine (O'Dell & Marshall, Neuroreport 1996 Nov. 4; 7(15-17):2457-61) induced degeneration

Models of Alzheimer's disease, such as: fimbria formix lesion model (Krugel et al., Int J Dev Neurosci 2001 June; 19(3):263-77), basal forebrain lesion model (Moyse E et al., Brain Res 1993 Apr. 2; 607(1-2):154-60).

Models of stroke, such as: Focal ischemia (Schwartz D A et al., Brain Res Mol Brain Res 2002 May 30; 101(1-2): 12-22); global ischemia (2- or 4-vessel occlusion) (Roof R L et al., Stroke 2001 November; 32(11):2648-57; Yagita Y et al., Stroke 2001 August; 32(8): 1890-6).

Models of multiple sclerosis, such as: myelin oligodendrocyte glycoprotein-induced experimental autoimmune encephalomyelitis (Slavin A et al., Autoimmunity 1998; 28(2): 109-20).

Models of amyotrophic lateral sclerosis, such as: pmn mouse model (Kennel P et al., J Neurol Sci 2000 Nov. 1; 180(1-2):55-61).

Models of anxiety, such as: elevated plus-maze test (Holmes A et al., Behav Neurosci 2001 October; 115(5):1129-44), marble burying test (Broekkamp et al., Eur J Pharmacol 1986 Jul. 31; 126(3):223-9), open field test (Pelleymounter et al., J Pharmacol Exp Ther 2002 July; 302(1):145-52).

Models of depression, such as: learned helplessness test, forced swim test (Shirayama Y et al., J Neurosci 2002 Apr. 15; 22(8):3251-61), bulbectomy (O'Connor et al., Prog Neuropsychopharmacol Biol Psychiatry 1988; 12(1):41-51).

Models for learning/memory, such as: Morris water maze test (Schenk F & Morris R G, Exp Brain Res 1985; 58(1): 11-28).

Models for Huntington's disease, such as: quinolinic acid injection (Marco S et al., J Neurobiol 2002 March; 50(4):323-32), transgenics/knock-ins (reviewed in Menalled L B and Chesselet M F, Trends Pharmacol Sci. 2002 January; 23(1):32-9).

Aged animals: using old mice/rats.

These models are contemplated with any particular adaptations needed for the method to be compliant with the compound administered, delivery system including formulation of the compound intended.

In certain embodiments of the invention, the effect of a stem and/or progenitor cell growth-modulating agent is tested in rats according to the following exemplary method:

Male rats can be used for testing the effect of a stem and/or progenitor cell growth-modulating agent on neurogenesis. If the experimental model animals are mice, a corresponding protocol can be used. The animals are housed in 12 hours light/dark regime, are fed with standard pellets; feeding and drinking is ad libitum; and 5 animals are housed per standard cage.

The stem and/or progenitor cell growth-modulating agent is administered by infusing the brain by osmotic mini-pumps for 1-14 days of BrdU or 3H-thymidine or other marker of proliferation, and relevant compound. Survival for 0-4 weeks post infusion.

Animal handling and surgery is performed as described in Pencea V et al., J. Neurosci Sep. 1 (2001), 21(17):6706-17, which is incorporated herein by reference in its entirety. The pumps are removed 1-14 days after insertion of the pump. The animals are anesthetized during the surgical procedures.

For the analysis of the effect of the stem and/or progenitor cell growth-modulating agent on neurogenesis, the animals are put under narcosis. Subsequently, transcardial perfusion with NaCl is performed. The animals are perfused with paraformaldehyde (4%) solution and decapitated. The brain is removed and fixated in paraformaldehyd (4%) solution over night. Subsequently, the brain is transfered into 30% sucrose solution at +4 C. The bulbus olfactorius (OB) is separated surgically, frozen inc −80° C. Methylbutan and stored in a −80° C. freezer.

The ipsilateral OB is sectioned in sagittal orientation and the rest of the brain is sectioned in coronal orientation on a cryotom.

Analysis and quantification is done for proliferative brain regions, migratory streams and areas of clinical relevance by immunohistochemistry.

One or several of the following antibodies can be used as markers: as neuronal markers NeuN, tubulin beta m, anti-tyrosine hydroxylase, anti-MAP-2 etc.; as glial markers anti-GFAP, anti-S 100 etc.; as oligodendrocyte markers anti-GaIC, anti-PLP etc. For BrdU visualisation: anti-BrdU. DAB (diamine benzidine) or fluorescence are used as labels for visualisation of the markers.

The results are quantified by, e.g., the following methods: For DAB-BrdU-Immunohistochemistry, stereological quantification in ipsilateral brain regions can be used.

The following regions of the brain can, inter alia, be evaluated: dorsal hippocampus dentate gyrus, dorsal hippocampus CA1/alveus, olfactory bulb (OB), subventricular zone (SVZ), and striatum.

In certain embodiments, quantification is conducted by double-staining and evaluation using a confocal laser microscope. For every structure (OB, DG, CA1/alveus, SVZ, wall-to-striatum) BrdU and a lineage marker are used as markers.

The experimental procedures are well-known to the skilled artisan and are described in detail in Pencea V et al., J. Neurosci Sep. 1 (2001), 21(17):6706-17, which is incorporated by reference herein in its entirety.

In certain embodiments, the effect of a stem and/or progenitor cell growth-modulating agent on self-renewal of stem cells or progenitor cells is tested in vivo in wild type animals or in CNS disease, disorder, trauma models. In certain embodiments, the effect of a stem and/or progenitor cell growth-modulating agent on differentiation of stem cells or progenitor cells is tested in vivo in wild type animals or in CNS disease, disorder, trauma models.

5.4.7 Screening Assays for Target Genes

In certain embodiments, the invention provides methods for the identification of genes whose expression is modulated in a stem cell by treatment, culturing, or maintaining of the stem cell with a stem and/or progenitor cell growth-modulating agent. In certain embodiments, the invention provides methods for the identification of genes whose expression is modulated in a neural stem cell by treatment, culturing, or maintaining of the neural stem cell with a stem and/or progenitor cell growth-modulating agent. In certain embodiments, the invention provides methods for the identification of genes whose expression is modulated in an adult stem cell by treatment, culturing, or maintaining of the adult stem cell with a stem and/or progenitor cell growth-modulating agent. In certain embodiments, the invention provides methods for the identification of genes whose expression is modulated in an embryonic stem cell by treatment, culturing, or maintaining of the embryonic stem cell with a stem and/or progenitor cell growth-modulating agent.

In certain embodiments, the invention provides methods for the identification of genes whose expression is modulated in a progenitor cell by treatment, culturing, or maintaining of the progenitor cell with a stem and/or progenitor cell growth-modulating agent. In certain embodiments, the invention provides methods for the identification of genes whose expression is modulated in a neural progenitor cell by treatment, culturing, or maintaining of the neural progenitor cell with a stem and/or progenitor cell growth-modulating agent. In certain embodiments, the invention provides methods for the identification of genes whose expression is modulated in an adult progenitor cell by treatment, culturing, or maintaining of the adult progenitor cell with a stem and/or progenitor cell growth-modulating agent. In certain embodiments, the invention provides methods for the identification of genes whose expression is modulated in an embryonic progenitor cell by treatment, culturing, or maintaining of the embryonic progenitor cell with a stem and/or progenitor cell growth-modulating agent.

Any technique well-known to the skilled art can be used to identify genes whose expression is upregulated or downregulated in a stem cell or progenitor cell in response to the treatment, culturing or maintaining of the stem cell or progenitor cell with a stem and/or progenitor cell growth-modulating agent. Such assays are useful, for example, for identifying proteins and genes involved in cellular proliferation and differentiation.

In certain embodiments, differential screening of cDNA libraries is used to identify the differentially expressed genes (Dulac and Axel, 1995, Cell 83:195-206). Briefly, RNA is isolated from cells that have been treated, maintained or cultured and from cells that have not been treated, maintained or cultured with a stem and/or progenitor cell growth-modulating agent. In a preferred embodiment, polyA mRNA is isolated. The RNA pools are labeled by any technique well-known to the skilled artisan. A cDNA library is generated from the cells. The library can be generated either from the cells that have been treated, cultured or maintained in the presence of the stem and/or progenitor cell growth-modulating agent or alternatively from the cells that have not been treated, cultured or maintained in the presence of the stem and/or progenitor cell growth-modulating agent. The library is plated and DNA of the different clones in the plated library is transferred to, e.g., nylon or nitrocellulose filters. Two identical set of lifts of the same library are taken. One set of filters is subsequently screened with the labeled RNA from the cells that have been treated, cultured or maintained with the stem and/or progenitor cell growth-modulating agent and the other set is hybridized with the other pool of labeled RNA from the cells that have not been treated, cultured or maintained with the stem and/or progenitor cell growth-modulating agent. Autoradiographs of the hybridized and washed two sets of filters are subsequently compared two each other. Signals that are either weaker or stronger on one set of the filters than on the other set of filters represent clones of candidate genes whose expression is modulated by treatment, culturing or maintaining cells in the presence of a stem and/or progenitor cell growth-modulating agent.

In certain embodiments, gene chips or microarrays of DNA are used to identify genes that are differentially regulated upon treatment, culturing or maintaining cells in the presence of a stem and/or progenitor cell growth-modulating agent (e.g., GeneChip®, Affymetrix, CA). Briefly, gene chips of DNA microarrays are prepared from the cells. Each DNA spot on the chip or array represents a particular gene. In a specific embodiment, chips or arrays of the cell type that is used are commercially available. RNA is isolated from cells that have been treated, maintained or cultured and from cells that have not been treated, maintained or cultured with a stem and/or progenitor cell growth-modulating agent. In a preferred embodiment, polyA mRNA is isolated. The RNA pools are labeled by any technique well-known to the skilled artisan. In a preferred embodiment, one RNA pool is labeled with a different fluorophore than the other RNA pool, such that the labels can be distinguished spectrophotometrically. Subsequently, the chip or array is hybridized with both, differentially labeled pools of RNA. The ratio of the strengths of the signals from one label to the other label can be evaluated using spectrophotometrical techniques. This ratio on any given DNA spot on the chip or array is representative of the relative quantities of the RNAs in the RNA pools. Thus, a ratio that deviates significantly from 1 for a particular DNA spot on the chip or array, i.e., for a particular gene, identifies that particular gene as a candidate for a gene that is differentially regulated upon treatment, culturing or maintaining the cells in the presence of a stem and/or progenitor cell growth-modulating agent.

Other techniques well-known to the skilled artisan to identify candidates of genes that are differentially regulated upon treatment, culturing or maintaining the cells in the presence of a stem and/or progenitor cell growth-modulating agent can be used with the methods of the invention. Such techniques include, but are not limited to SAGE (Velculescu, 1995, Science 270:484-487).

In certain embodiments, Northern blots or Western blots are conducted to identify genes that are differentially regulated upon treatment, culturing or maintaining the cells in the presence of a stem and/or progenitor cell growth-modulating agent. In specific embodiments, Northern blots of RNA from cells that have been treated, cultured or maintained in the presence of a stem and/or progenitor cell growth-modulating agent and Northern blots of RNA from cells that have not been treated, cultured or maintained are hybridized with probes that are specific for an immediate early gene, such as, but not limited to, but not limited to, c-fos, c-jun, c-myc, jun-B, fos-B, SRF (serum response factor), arc, egr-1.

In certain embodiments, the time period of treating, culturing or maintaining is at least 1 hour, 2 hours, 4 hours, 6 hours, 8 hours, 12 hours, 18 hours, 1 day, 1.5 days, 2 days, 2.5 days, 3 days, 3.5 days, 4 days, 4.5 days, 5 days, 5.5 days, 6 days, 6.5 days, 1 week, 2 weeks, 3 weeks, 1 month, 2 months, 3 months, 4 months, 5 months, 6 months, 9 months, 1 year, 1.5 years, or at least 2 years. In certain embodiments, the time period of treating, culturing or maintaining is at most 1 hour, 2 hours, 4 hours, 6 hours, 8 hours, 12 hours, 18 hours, 1 day, 1.5 days, 2 days, 2.5 days, 3 days, 3.5 days, 4 days, 4.5 days, 5 days, 5.5 days, 6 days, 6.5 days, 1 week, 2 weeks, 3 weeks, 1 month, 2 months, 3 months, 4 months, 5 months, 6 months, 9 months, 1 year, 1.5 years, or at most 2 years.

5.4.8 Screening Assays for Target Gene Activities

In certain embodiments, the invention provides methods for the identification of genes whose activity is modulated in a stem cell by treatment, culturing, or maintaining of the stem cell with a stem and/or progenitor cell growth-modulating agent. In certain embodiments, the invention provides methods for the identification of genes whose activity is modulated in a neural stem cell by treatment, culturing, or maintaining of the neural stem cell with a stem and/or progenitor cell growth-modulating agent. In certain embodiments, the invention provides methods for the identification of genes whose activity is modulated in an adult stem cell by treatment, culturing, or maintaining of the adult stem cell with a stem and/or progenitor cell growth-modulating agent. In certain embodiments, the invention provides methods for the identification of genes whose activity is modulated in an embryonic stem cell by treatment, culturing, or maintaining of the embryonic stem cell with a stem and/or progenitor cell growth-modulating agent.

In certain embodiments, the invention provides methods for the identification of genes whose activity is modulated in a progenitor cell by treatment, culturing, or maintaining of the progenitor cell with a stem and/or progenitor cell growth-modulating agent. In certain embodiments, the invention provides methods for the identification of genes whose activity is modulated in a neural progenitor cell by treatment, culturing, or maintaining of the neural progenitor cell with a stem and/or progenitor cell growth-modulating agent. In certain embodiments, the invention provides methods for the identification of genes whose activity is modulated in an adult progenitor cell by treatment, culturing, or maintaining of the adult progenitor cell with a stem and/or progenitor cell growth-modulating agent. In certain embodiments, the invention provides methods for the identification of genes whose activity is modulated in an embryonic progenitor cell by treatment, culturing, or maintaining of the embryonic progenitor cell with a stem and/or progenitor cell growth-modulating agent.

In certain embodiments, a suicide inhibitor of a particular class of enzymes, i.e., proteins with a common biochemical activity, is labeled with a biotin molecule. In a specific embodiment, a suicide inhibitor of proteases is labeled with a biotin molecule. Protein extracts are prepared from cells that have been treated, cultured or maintained in the presence of a stem and/or progenitor cell growth-modulating agent. Protein extracts are prepared from cells that have not been treated, cultured or maintained in the presence of a stem and/or progenitor cell growth-modulating agent. The different protein extracts are then separately incubated with the labeled suicide inhibitor for a certain time period. Only active enzymes will irreversibly bind to the inhibitor-biotin complex. Subsequently, the active proteins that are bound to the inhibitor-biotin complex are purified from the extracts and are resolved on a SDS PAGE. The amounts of the proteins are representative of the activity of the enzymes in the cell. Different amounts between the two cell populations identify candidates of enzymes that are differentially regulated upon treatment, culturing, or maintaining the cells in the presence of a stem and/or progenitor cell growth-modulating agent.

5.4.9 Drug Screening Assays

In certain embodiments, the present invention relates to methods of using the cell that has been treated, cultured or maintained in the presence of a stem and/or progenitor cell growth-modulating agent for drug-screening assays. In certain embodiments, the cells of the invention are used for in vitro drug-screening assays. In specific embodiments, stimulating or inhibiting activity of a drug candidate on stem cell or progenitor cell proliferation or differentiation into particular neuronal phenotype or glial subtype is tested. In a particular embodiment, the invention relates to a method of screening for differentiation inducing agents, the method comprising culturing stem cells or progenitor cells in the presence of a stem and/or progenitor cell growth-modulating agent, exposing the cells to one or more candidate agents, and assaying for one or more indicators of differentiation. In certain embodiments, the cells are first incubated in the presence of a stem and/or progenitor cell growth-modulating agent and subsequently tested with the candidate drug. Screening for inhibitors of differentiation can be performed by, for example, culturing stem cells or progenitor cells with a stem and/or progenitor cell growth-modulating agent, exposing the cells to one or more known differentiating agents, and either before, concomitantly with, or after exposure to the differentiating agent or agents, exposing the cells to one or more candidate inhibitors of differentiation and assaying for one or more indicators of differentiation.

When screening for candidate proliferation inducing agents, the method may, e.g., involve culturing of stem cells or progenitor cells in the presence of a stem and/or progenitor cell growth-modulating agent, exposing the cells to one or more candidate proliferation inducing agents, and assaying for enhanced neural stem cell growth. Screening for inhibitors of proliferation can be performed by, for example, culturing stem cells or progenitor cells in the presence of a stem and/or progenitor cell growth-modulating agent, optionally exposing the cells to one or more known proliferation inducing agents, and either before, concomitantly with, or after exposure to the proliferation inducing agent or agents, if used, exposing the cells to one or more candidate inhibitors of proliferation and assaying for one or more indicators of proliferation. The present invention also relates to the substances obtained by the methods defined above.

In other embodiments, the present invention relates to an unbiased quantitative or qualitative, preferably quantitative, method to assess neurogenesis and migratory streams of neural stem cell or neural progenitor progeny that have been cultured or are being cultured in the presence of a stem and/or progenitor cell growth-modulating agent, preferably in in vivo assays, in various regions of the brain as well as techniques to analyze the total number of stem cells and their progeny migrating to various regions of the brain. This is for the development of new screening methods, which methods are also within the scope of the present invention as defined by the appended claims. This could be of use in diagnosing patients suffering from neurodegenerative diseases, if the development of ependymal cell markers suitable for positron emission tomography (PET), or other imaging systems able to visualize the living brain with sufficient resolution, allows the diagnosis of defective migration and/or differentiation of the stem cell progeny in human CNS.

5.5 Cell Types for Use with the Invention

In certain embodiments of the invention, the cell that is to be cultured using the methods of the invention is a stem cell. In certain embodiments of the invention, the cell that is to be cultured using the methods of the invention is a fetal stem cell. In certain embodiments of the invention, the cell that is to be cultured using the methods of the invention is an adult stem cell. In certain embodiments of the invention, the cell that is to be cultured using the methods of the invention is a neural stem cell. In certain embodiments of the invention, the cell that is to be cultured using the methods of the invention is a fetal neural stem cell. In certain embodiments of the invention, the cell that is to be cultured using the methods of the invention is an adult neural stem cell. In certain embodiments of the invention, the cell that is to be cultured using the methods of the invention is an embryonal stem cell. In certain embodiments of the invention, the cell that is to be cultured using the methods of the invention is a progenitor cell. In certain embodiments of the invention, the cell that is to be cultured using the methods of the invention is a fetal progenitor cell. In certain embodiments of the invention, the cell that is to be cultured using the methods of the invention is an adult progenitor cell. In certain embodiments of the invention, the cell that is to be cultured using the methods of the invention is a neural progenitor cell. In certain embodiments of the invention, the cell that is to be cultured using the methods of the invention is a fetal neural progenitor cell. In certain embodiments of the invention, the cell that is to be cultured using the methods of the invention is an adult neural progenitor cell.

Without being bound by theory, in the context of the present invention, a stem cell is a cell that is capable of undergoing self-renewal. Further, without being bound by theory, in the context of the present invention, a progenitor cell is not capable of self-renewal.

In a certain specific embodiment, the cell to be cultured using the methods of the invention is not a cell of a hematopoietic cell line. In a more specific embodiment, the cell to be cultured using the methods of the invention is not an erythroid burst-forming unit. As used in the context of the present invention, stem cells include, but are not limited to, stem cells of endothelial, mesenchymal, epithelial, haemopoietic, pancreatic, and muscular origins. Stem cells of endoderm origin include, but are not limited to, stem cells of gut, pancreas, and liver. Stem cells of ectoderm origin include, but are not limited to, stem cells of epidermal tissue and the nervous system. Stem cells of Mesoderm origin include, but are not limited to, muscle, bone, and blood.

5.5.1 Isolating Ependymal Neural CNS Stem Cells

In a specific embodiment of the invention, the stem cell to be cultured or maintained by the methods of the invention is an ependymal neural CNS stem cells. An ependymal neural CNS stem cell can be obtained by the methods described below (Johansson et al., 1999, Cell 96:25-34).

The animal from which the cells are isolated may be an animal or a human. In certain embodiments, the ependymal cells are from tissue comprising the walls of the ventricular system of the brain or spinal cord, or any other area that contains ependymal cells. The dissection and recovery of such tissue is easily performed by the skilled man in this field by any suitable routine method. The dissociation of the tissue into individual cells is performed by any suitable method, such as an enzymatic and/or mechanical treatment, and is not restricted in any way as long as the desired single cells are obtained as a result thereof. Examples of such methods are, e.g., trituration, trypsin treatment, collagenase treatment and hyaluronidase treatment. Most preferably, the dissociation is performed by enzymatic treatment with trypsin. The dissociation of tissue may alternatively be performed by any other method easily chosen by the skilled artisan in view of the prevailing conditions.

The screening of the resulting cells is also performed by any suitable method depending on the characteristic, trait or property of an ependymal cell used. In one embodiment of the present method, the screening is performed by use of the expression of a specific cell surface marker, such as a protein. Such an expression of a surface protein may for example be the expression of the Notch1 receptor. In an alternative embodiment of this aspect of the invention, the single cells are screened for by specifically labeling ependymal neural stem cells or ependymal cells and choosing so labeled cells. Such a labeling may be a dye and is advantageously a fluorescent labeling, such as DiI. However, in an alternative embodiment a virus, such as an adenovirus, may be used to label the cells. The labeling of cells is used extensively within research and diagnostic methods and the choice of a suitable technique is thus easily within the skill of one in the art.

In a preferred embodiment of the method according to the invention, the cells recovered are comprised of at least about 10% of ependymal neural stem cells, such as 10-50%, e.g., about 35%, or in a preferred embodiment, up to about 90%, or most preferably an essentially pure culture of ependymal neural stem cells.

In a specific embodiment, the ependymal stem cells are isolated in the following manner:

The lateral wall of the lateral ventricles and the spinal cord are enzymatically dissociated in 0.7 mg/ml hyaluronic acid, 0.2 mg/ml kynurenic acid, and 1.33 mg/ml trypsin in HBSS with 2 mM glucose at 37° C. for 30 min. The cells are centrifuged at 200 g for 5 min, resuspended in 0.9 M sucrose in 0.5×HBSS, and centrifuged for 10 min at 750 g. The cell pellet is resuspended in 2 ml of culture medium, placed on top of 10 ml 4% BSA in EDSS solution, and centrifuged at 200 g for 7 min, followed by washing in DMEM/F12. The culture medium consists of 10 ng/ml bFGF, B27 supplement, 2 mM glutamin, 100 U/ml penicillin, and 100 μg/ml streptomycin in DMEM-F-12 medium. Single cells were cultrured in 96-well dished in 50% neurophere-conditioned medium and 50% fresh medium.

The dissociation solution consisted of 0.075% collagenase type 1, 0.075% hyaluronidase, and 500 U/ml DNasel in 0.2 M PIPES. The cells were resuspended in 1:100 anti-Notch 1 antiserum (Mitsiadis et al., 1995) and incubated for 20 min at 4° C. After rising in a large volume of DMEM/F12, the cells were resuspended in 100 μl, of culture medium containing magnetic bead-conjugated goat anti-rabbit antiserum (1.8-2.1×10⁷ beads/100 μl, Dynal) and were incubated for 20 min at 4° C. Subsequently, 2 ml of culture medium was added to the tube that was placed in the magnetic separator. After 2 min, the supernatant containing cells that had not bound magnetic beads was collected at 2 ml culture medium was added to the tube. The magnet was then removed and the cells that had bound magnetic beads were collected.

Copending and co-owned U.S. patent application Ser. No. 09/104,772, filed Jun. 25, 1998, entitled “Method of Isolating Ependymal Neural Stem Cells”, by Frisen et al., and U.S. patent application Ser. No. 09/719,001, filed Jul. 12, 2001, entitled “Ependymal Neural Stem Cells and Methods for Their Isolation”, by Janson et al. are incorporated herein by reference in their entireties for all purposes, including the description of the methods of isolating and using ependymal neural stem cells.

5.5.2 Genetically Modified Cells

In certain embodiments, the present invention relates to culturing, maintaining, and treating genetically modified stem cells or pregenitor cells in the presence of a stem and/or progenitor cell growth-modulating agent. In certain more specific embodiments, the present invention relates to culturing maintaining, and treating genetically modified neural stem cells or neural pregenitor cells in the presence of a stem and/or progenitor cell growth-modulating agent.

Manipulations may be performed in order to modify various properties of the cell, e.g., to render it more adapted or resistant to certain environmental conditions, to induce a production of one or more certain substances therefrom, which substances may e.g. improve the viability of the cell or alternatively may be useful as drugs or medicaments. The invention of methods to purify ependymal neural stem cells in cell culture allows for all types of genetic manipulation, for example transfection of these cells with plasmid or viral expression vectors or purification of cells from transgenic organisms or suppression of gene expression with for example antisense DNA or RNA fragments. Localization of the ependymal neural stem cell in vivo allows for alteration of gene expression in these cells in situ with for example viral vectors.

In certain embodiments, the cells are obtained from genetically manipulated animal. In a specific embodiment, the cells are obtained from a genetically modified murine disease model. In a more specific embodiment, the cells are obtained from a murine model for a neurodegenerative disorder.

In certain embodiments, the cells are taken from a transgenic organism. Transgenic animals include, but are not limited to, “knock out” animals, animals ectopically expressing genes or fragments of genes, animals over-expressing genes or fragments of genes, animals expressing antisense RNA fragments. Under certain conditions, it may be valuable to use cells which lack a certain gene or produces lower levels of the gene product. In specific embodiments, the gene that is not expressed or expressed at lower levels in the cells is a cell surface molecule. In specific embodiments, the gene that is not expressed or expressed at lower levels in the cells is an immunogenic molecule.

In specific embodiments, the cells are obtained from a PTPase “knock out” mouse.

In certain embodiments, the cells are obtained from animal model of a CNS disorder, disease, or trauma (see 5.4.6).

5.6 Pharmaceutical Methods and Compositions

In certain embodiments, the invention relates to methods of treating and/or preventing stem cell related diseases and/or disorders. The invention further relates to progenitor cell related diseases and/or disorders. In particular, the invention relates to disease and disorders involving a deficiency in stem and/or progenitor cells. In specific embodiments, the methods of the invention relate to treating and/or preventing diseases and/or disorders involving a deficiency in healthy stem and/or progenitor cells. In certain specific embodiments, the methods of the invention relate to treating and/or preventing diseases and/or disorders involving malfunctioning stem and/or progenitor cells. In even more specific embodiments, the invention relates to a deficiency in neural stem cells.

The diseases and disorders to be treated by the methods of the invention include, but are not limited to, Parkinson's Disease, Alzheimer's Disease, Amyotrophic Lateral Sclerosis, Multiple Sclerosis, Spinal Cord Injury (as caused by, e.g., infection, inflammation, trauma, cancer, osteoporosis), Stroke, Depression, Drug abuse, diseases and/or disorder affecting memory. In specific embodiments, the methods of the invention include treating and/or preventing ageing deficits and obesity. In certain embodiments, the invention provides methods for regulating eating behaviour. In certain embodiments, the methods of the invention relate to the treatment and/or prevention of CNS diseases.

In certain embodiments, the methods of treating and/or preventing comprise administering to a subject in need of treatment a stem cell and/or a progenitor cell that has been treated, propagated, cultured, or maintained in the presence of a stem and/or progenitor cell growth-modulating agent. In certain, more specific, embodiments, the methods of treating and/or preventing comprise administering to a subject in need of treatment a stem cell and/or a progenitor cell that has been treated, propagated, cultured, or maintained in the presence of a phosphate mimic.

In certain other embodiments, the methods of treating and/or preventing comprise administering to a subject in need of treatment a stem and/or progenitor cell growth-modulating agent. In certain, more specific, embodiments, the methods of treating and/or preventing comprise administering to a subject in need of treatment a phosphate mimic.

The subject can be, e.g., a mouse, a rat, a dog, or a primate. Most preferably, the subject is a human.

As used herein, “cells of the invention” refer collectively to stem cells or progenitor cells that have been treated, maintained, or cultured in the presence of a stem and/or progenitor cell growth-modulating agent. Further, “cells of the invention” refer to neural stem cells, neural progenitor cells, adult neural stem cells, adult neural progenitor cells, embryonic neural stem cells, embryonic neural progenitor cells, adult stem cells, adult progenitor cells, embryonic stem cells, or embryonic progenitor cells that have been treated, maintained, or cultured in the presence of a stem and/or progenitor cell growth-modulating agent.

In certain embodiments, the present invention relates to stem cell or progenitor cell that has been treated, maintained, or cultured in the presence of a stem and/or progenitor cell growth-modulating agent for use in therapy, e.g., as a medicament. In addition, the invention also relates to the use of cells that has been treated, maintained, or cultured in the presence of a stem and/or progenitor cell growth-modulating agent in the preparation of a medicament for regulating the neurogenesis or gliogenesis in the central nervous system, such as the brain. Such regulation is either inducing or inhibiting and the treatment may be aimed at Parkinson's disease, Alzheimer's disease, stroke, trauma etc. In the case of glial cells, the medicament may be intended for treating multiple sclerosis and other glia related conditions. In one particular embodiment of the invention, these aspects of the invention use ependymal neural stem cells obtained by the method discribed above.

In certain embodiments, the invention also encompasses the uses of other stem cells that has been treated, maintained, or cultured in the presence of a stem and/or progenitor cell growth-modulating agent for therapy or prevention of certain stem cell-related disorders.

In a further aspect, the invention relates to a pharmaceutical preparation comprising one or more stem cells and/or progenitor cells that have been treated, maintained, or cultured in the presence of a stem and/or progenitor cell growth-modulating agent according to the invention and a pharmaceutically acceptable carrier. In a further aspect, the invention relates to a pharmaceutical preparation comprising one or more neural stem cells and/or neural progenitor cells that have been treated, maintained, or cultured in the presence of a stem and/or progenitor cell growth-modulating agent according to the invention and a pharmaceutically acceptable carrier.

In a further aspect, the invention relates to a pharmaceutical preparation comprising one or more stem cells and/or progenitor cells that have been treated, maintained, or cultured in the presence of a stem and/or progenitor cell growth-modulating agent according to the invention, pharmaceutically acceptable carrier, and a stem and/or progenitor cell growth-modulating agent. In a further aspect, the invention relates to a pharmaceutical preparation comprising one or more neural stem cells and/or neural progenitor cells that have been treated, maintained, or cultured in the presence of a stem and/or progenitor cell growth-modulating agent according to the invention, a stem and/or progenitor cell growth-modulating agent, and a pharmaceutically acceptable carrier.

The preparations according to the invention may be adapted for injection into a suitable part of the central nervous system. Such a pharmaceutical preparation comprises any suitable carrier, such as an aqueous carrier, e.g. buffered saline etc. The active composition of the present preparation is generally sterile and free of any undesirable matter. In addition, the preparations may contain pharmaceutically acceptable auxiliary substances as required to approximate physiological conditions, such as pH adjusting agents etc. The concentration of the stem cell or progenitor cell that has been treated, cultured or maintained in the presence of a stem and/or progenitor cell growth-modulating agent in the preparation will vary depending on the intended application thereof and the dosages thereof are decided accordingly by the patient's physician. The pharmaceutical compositions of the present invention comprise about 10³ to 10⁹ cells of the invention. In some preferred embodiments, the compositions comprise about 10⁵ to 10⁸ cells of the invention. In some preferred embodiments, the compositions comprise about 10⁷ cells of the invention. The cells used may have been isolated by the method discribed above or any other suitable method or obtained in any other way. In a specific embodiment, the stem cell or progenitor cell may have been genetically manipulated in order to be especially adapted for the intended use. In a further aspect, the present invention also relates to an animal, such as a mouse, that comprises a genetically modified stem cell or progenitor cell that has been treated, cultured or maintained in the presence of a stem and/or progenitor cell growth-modulating agent according to the invention. Such animals may, e.g., be useful as models in research or for the testing of drugs.

In certain embodiments, the present invention relates to a method of treating a subject afflicted with a neurodegenerative disease, which method comprises the administration to said subject of a pharmaceutically effective amount of neural stem cells or progenitor cells that have been cultured, treated, or maintained in the presence of a stem and/or progenitor cell growth-modulating agent. In certain embodiments, neural stem cells or progenitor cells are co-administered with a stem and/or progenitor cell growth-modulating agent to the subject. The subject may be any animal, including a human. There are several potential injection sites. Thus, the cells could be injected into the nerve terminal area of the cells that degenerate in the particular neurodegenerative disorder. For example, in Parkinson's Disease, the dopamine neurons that die are situated in the midbrain in substantia nigra pars compacta, but the cells can be transplanted into the nerve terminal area in the forebrain. Alternatively, they may be transplanted directly into the ventricular system, into the migratory streams of cells described in the examples below, or in the neuronal cell body region of the cells that degenerate in the particular human neurodegnerative disorder. In general, such a method is based on administration of stem cells with an unimpaired function and ability to produce neurons or other cell types depending on the human CNS disorder. Alternatively, neurons or glial cells generated from stem cells in vitro can be administrated to the CNS. Methods for transplanting cells into the brain have been described, and are known to one of skill in the art (Widner, et al., New England J. Med., 327:1556; Wenning, et al., 1997, Ann. Neurol., 42(1):95-107; Lindvall, et al., 1994, Ann. Neurol., 35(2):172-80; Widner, et al., 1993, Acta Neurol Scand Suppl, 146:43-5; Neural Grafting in the Mammalian CNS, 1985, Bjorklund and Stenevi, eds; U.S. Pat. No. 5,650,148; International Patent Publication WO 9206702, Itukura, T., et al., 1988, J. Neurosurg. 68:955-959, each of which are incorporated herein by reference).

In an alternative embodiment, the invention relates to a method of treatment and/or prevention of neurodegenerative disorders in a human or animal subject, wherein the existing defective neural stem cells' ability to produce new neurons or migrate to the appropriate target is restored. Such a method is based on the administration of a stem and/or progenitor cell growth-modulating agent.

In certain embodiments, the invention provides methods that comprise a regimen of administering cells of the invention and a regiment of administering a stem and/or progenitor cell growth-modulating agent. In certain embodiments, the stem and/or progenitor cell growth-modulating agent is administered a certain time period before administering cells of the invention. In other embodiments, the cells of the invention are administered a certain time period prior to administering a stem and/or progenitor cell growth-modulating agent. In certain embodiments, the cells of the invention are administered repeatedly, each administration a certain time period separated from the other administrations. In certain embodiments, the cells of the invention are administered in a sequence, each administration of the sequence is a certain time period separated from the other administrations, and a stem and/or progenitor cell growth-modulating agent is administered prior to, concurrently with, or subsequently to administering the cells of the invention.

In summary, the present invention will make it possible to develop new treatment strategies in diverse diseases of the CNS, not only in diseases with a slow progression of the neurodegeneration (including Alzheimer's disease, Parkinson's disease, amyotrophic lateral sclerosis, multiple sclerosis) but also in clinical situations of acute trauma to the head or spinal cord as well as in cerebrovascular diseases.

5.6.1 Reinfusion of Stem Cells

The stem cells or progenitor cells that were cultured, propagated, treated, or maintained using the methods of the invention are reinfused into the subject systemically, preferably intradermally, by conventional clinical procedures. These activated cells are reinfused, preferentially by systemic administration into the autologous patient. The subject is preferably an animal, including, but not limited, to an animal such as a cow, horse, sheep, pig, chicken, turkey, quail, cat, dog, mouse, rat, rabbit, guinea pig, etc., and is more preferably a mammal, and most preferably a human.

5.6.2 Effective Dose

The compositions of the present invention, comprising an effective amount of stem cells or progenitor cells that have been treated, maintained or cultured in the presence of a stem and/or progenitor cell growth-modulating agent are administered to a subject in need of treatment. In certain other embodiments, the compositions of the present invention, comprising an effective amount of stem cells or progenitor cells that have been treated, maintained or cultured in the presence of a stem and/or progenitor cell growth-modulating agent and a stem and/or progenitor cell growth-modulating agent are administered to a subject in need of treatment. Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. While compositions that exhibit toxic side effects may be used, care should be taken to design a delivery system that targets such complexes to the site of affected tissue in order to minimize potential damage to unaffected cells and, thereby, reduce side effects.

In one embodiment, the data obtained from the cell culture assays and animal studies can be used in formulating a range of dosage for use in humans. The dosage of compositions lies preferably within a range of circulating concentrations that include the ED₅₀ with little or no toxicity. The dosage may vary within this range depending upon the dosage form employed and the route of administration utilized. For any complexes used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.6.3 Formulations and Use

Pharmaceutical compositions for use in accordance with the present invention may be formulated in conventional manner using one or more physiologically acceptable carriers or excipients.

Thus, the cells and stem and/or progenitor cell growth-modulating agents and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose), fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the cells of the invention. In certain embodiments, controlled release devices are used for the administration of the cells of the invention and a stem and/or progenitor cell growth-modulating agent to a subject.

For buccal administration the compositions may take the form of tablets or lozenges formulated in conventional manner.

The cells of the invention may be formulated for parenteral administration by injection, e.g., by bolus injection or continuous infusion. In certain embodiments, the cells of the invention are administered with a stem and/or progenitor cell growth-modulating agent. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The stem and/or progenitor cell growth-modulating agent can be administered by any technique well-known to the skilled artisan.

The cells of the invention and/or the stem and/or progenitor cell growth-modulating agent may also be formulated in rectal compositions such as suppositories or retention enemas, e.g., containing conventional suppository bases such as cocoa butter or other glycerides.

In addition to the formulations described previously, the cells of the invention and/or the stem and/or progenitor cell growth-modulating agent may also be formulated as a depot preparation. Such long acting formulations may be administered by implantation (for example subcutaneously or intramuscularly) or by intramuscular injection. Thus, for example, the cells of the invention and/or the stem and/or progenitor cell growth-modulating agent may be formulated with suitable polymeric or hydrophobic materials (for example as an emulsion in an acceptable oil) or ion exchange resins, or as sparingly soluble derivatives, for example, as a sparingly soluble salt.

The cells of the invention and/or the stem and/or progenitor cell growth-modulating agent may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Stem and/or progenitor cell growth-promoting agents may be designed to pass across the blood brain barrier (BBB). For example, a carrier such as a fatty acid, inositol or cholesterol may be selected that is able to penetrate the BBB. In certain embodiments, cyclodextrin can be used as a carrier to transport stem and/or progenitor cell growth-promoting agents across the blood brain barrier. The carrier may be a substance that enters the brain through a specific transport system in brain endothelial cells. The carrier may be coupled to the active agent or may contain/be in admixture with the active agent. The carrier may be covalently linker to the active agent. In other embodiments, the carrier and the active agent are coupled to each other by caging or chelating. Liposomes can be used to cross the BBB. WO911 04014 describes a liposome delivery system in which an active agent can be encapsulated/embedded and in which molecules that are normally transported across the BBB are present on the liposome outer surface. Liposome delivery systems are also discussed in U.S. Pat. No. 4,704,355. In certain embodiments, the invention relates to methods using bioisosteres of stem and/or progenitor cell growth-promoting agents.

Stem and/or progenitor cell growth-promoting agents can be formulated (uptake enhancing agent added but not chemically bound) to- or chemically modified (conjugated with uptake enhacing agent) to become cell-permeable, blood-brain barrier permeable, choroid plexus-brain permeable, (eg nasal-, rectal-) mucosa-blood permeable. Uptake enhancing agents include, but are not limited to, pore forming agents and agents that disrupt cell-cell-junctions (eg taurocholate), or agents that cage/chelate (eg cyclodextrins). Chemical modifications that promote uptake of an agent by a cell include substituents (often peptides, sugars and proteins) that mediate cell uptake via membrane-bound carriers (pumps, receptors, channels) or other substituents that mediate cell uptake via more nonspecific mechanisms (eg certain peptidic compounds).

The stem and/or progenitor cell growth-promoting agents and/or the cells of the invention can also be administered through intracerebroventricular- or intraparenchymal (in brain) infusion. We want to specifically exemplify/claim these parenteral routes as they are of importance to us.

All techniques for the delivery of agents and/or cells well-known to the skilled artisan can be used with the methods and compositions of the invention.

### 6. EXAMPLES

Materials & Methods

Neurosphere cultures: The anterior lateral wall of the lateral ventricle of 5-6 week old mice was enzymatically dissociated in 0.8 mg/ml hyaluronidase and 0.5 mg/ml trypsin in DMEM containing 4.5 mg/ml glucose and 80 units/ml DNase at 37° C. for 20 min. The cells were gently triturated and mixed with three volumes of Neurosphere medium (DMEM/F12, B27 supplement, 125 mM HEPES pH7.4) containing 20 ng/ml EGF (unless otherwise stated), 100 units/ml penicillin and 100 _g/ml streptomycin. After passing through a 70 _m strainer, the cells were pelleted at 160×g for 5 min. The supernatant was subsequently removed and the cells resuspended in Neurosphere medium supplemented as above, plated out in culture dishes and incubated at 37° C. Neurospheres were ready to be split approximately 7 days after plating.

To split the neurospheres, they were collected by centrifugation at 160×g for 5 min. The conditioned supernatant (conditioned medium) was removed and saved. The neurospheres were resuspended in 0.5 ml Trypsin/EDTA in HBSS (1×), incubated at 37° C. for 2 min and triturated gently to aid dissociation. Following a further 3 min incubation at 37° C. and trituration, 3 volumes of ice cold NSPH-media-EGF were added to stop further trypsin activity. The cells were pelleted at 220×g for 4 min, and resuspended in a 1:1 mixture of fresh Neurosphere medium and conditioned medium. EGF was supplemented to 20 ng/ml and the culture plated out and incubated at 37° C.

Where stated, a minimal Neurosphere medium was used containing BIT supplement in place of B27 supplement.

Neurosphere assays: NSC, cultured as described above, from passage 2 were seeded in DMEM/F12 supplemented with B27 into a 96-well plate as single cells (10000 cells/well), to which agents were added at the concentrations indicated.

Intracellular ATP assay: Intracellular ATP levels have previously been shown to correlate to cell number (Crouch, Kozlowski et al., 1993, J Immunol Methods 160(1):81-8). After 3 days of treatment, intracelluar ATP was measured using the ViaLight kit (BioWhittaker) according to the manufacturer's instructions.

Culture of human neural stem cells: Human neural stem cells were isolated and cultured under adherent conditions according to the protocol described in Palmer et al., 2001, Nature 411(6833):42-43, modified to exclude the additives PDGF and cystatinC and using B27 supplement instead of BIT. The cells were cultured for 10-16 passages in DMEM/F12 with B27-supplement and EGF (20 ng/ml) and FGF-2 (20 ng/ml).

Cells were detached with Trypsin/EDTA, rinsed three times and then replated in suspension culture plastic wells in DMEM/F12 and B27-supplement for 24 h without growth factors. Thereafter factors (Vanadate, EGF or EGF+FGF2) were added for one day.

Results

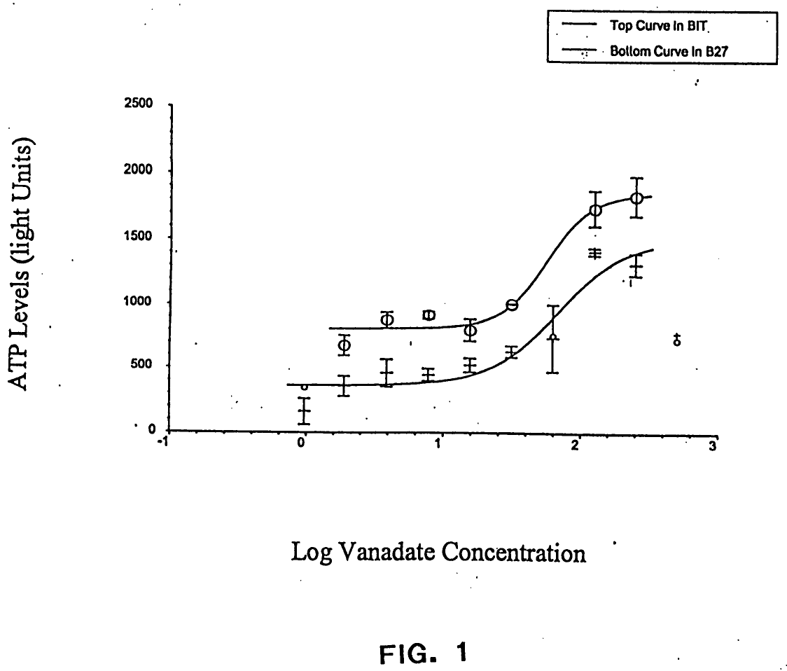

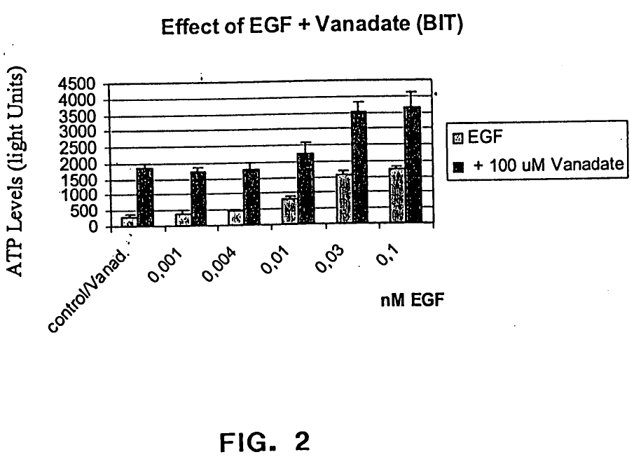

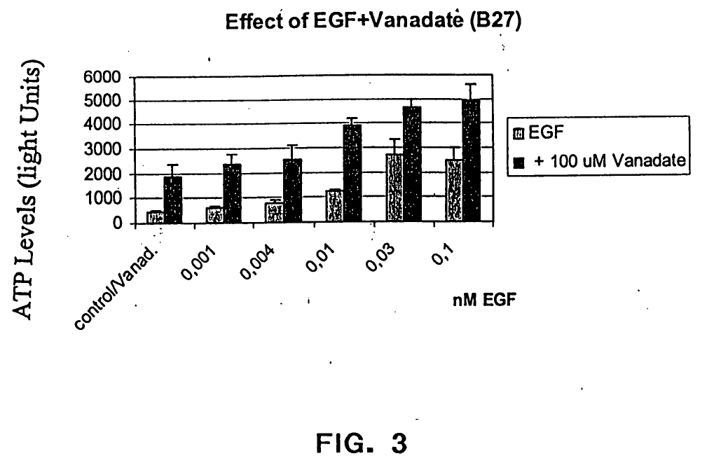

In growth media supplemented with either BIT (growth cocktail containing BSA, insulin and transferrin) or the richer supplement B27 (many factors including the above), vanadate had a concentration dependent proliferative effect when added alone (FIG. 1). This effect was additive to a to the effect of EGF. The maximal amplitudes of responses to the two compounds added alone was equal in size (FIGS. 2 and 3).

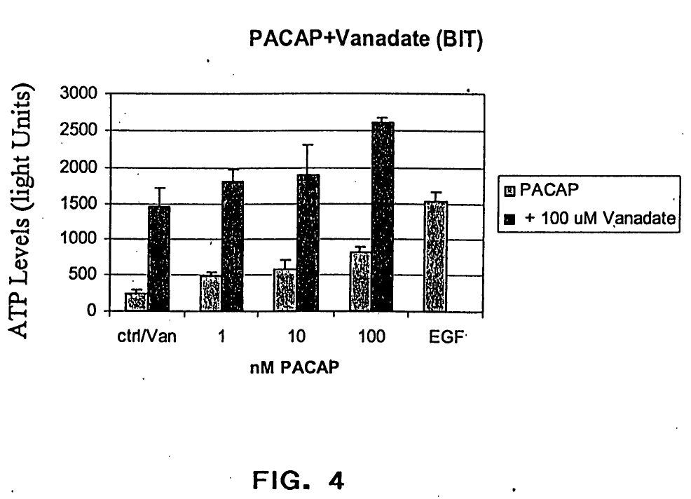

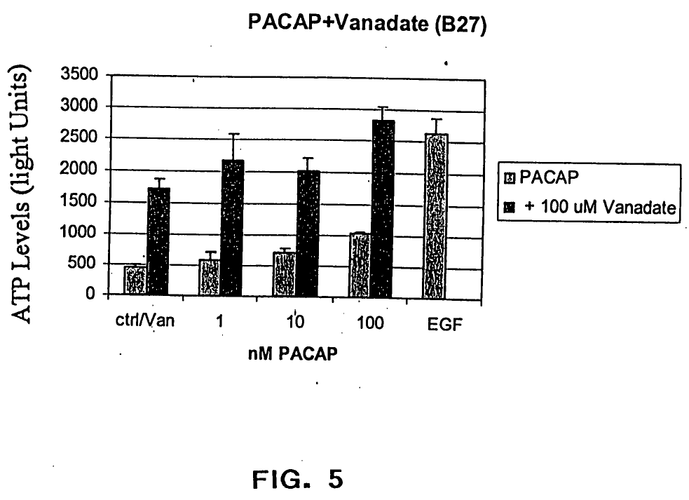

In adult derived neural stem cells, increased levels of cAMP trigger proliferation. Our unpublished results show that direct adenylate cyclase stimulation (eg forskolin), cyclase activating GPCRs (exemplified by responses to neuropeptide PACAP) and inhibition by phosphodiesterase IV (eg rolipram) all trigger proliferation. The effect of vanadate is additive to the effect of PACAP (FIGS. 4, 5).

Sphere Formation in Presence of Vanadate and in Combination with PACAP

Cells were isolated as described (see section 5.5.1). Aliquots of 1 ml cell suspension per well equivalent to one brain per well were plated in DMEM/F12 supplemented with B27.

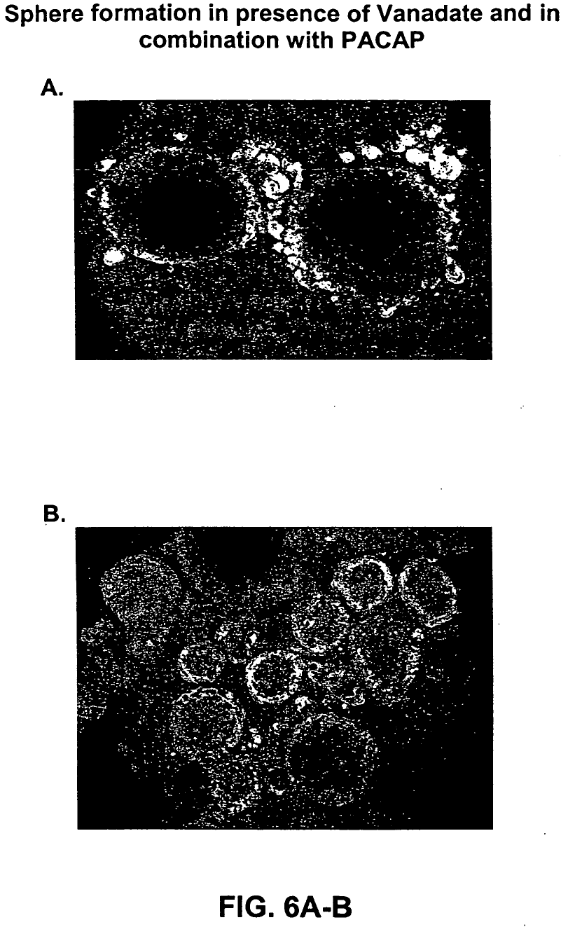

10 uM Vanadate or 10 uM Vanadate +300 nM PACAP was added to the cultures. Fresh substances were added every second day. After 7 days sphereformation was observed. Sphereforming capacity was noted in cultures receiving Vanadate alone and in cultures receiving Vanadate and PACAP (FIG. 6).

Vanadate in Combination with EGF

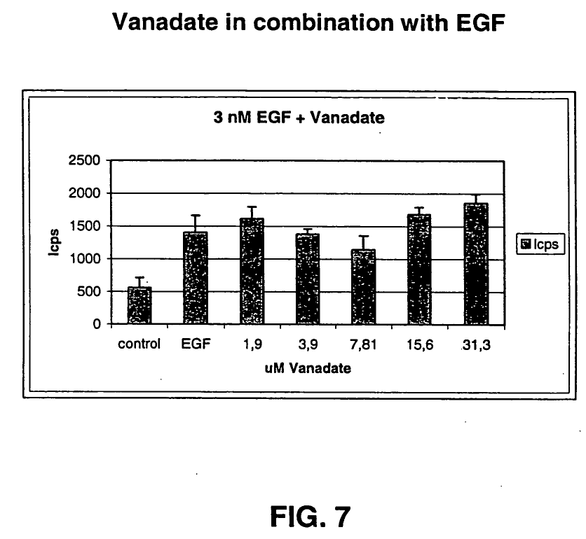

Cells were isolated and cultured as described previously (see section 5.5.1). ATP-levels were measured as described previously (Crouch, Kozlowski et al., 1993, J Immunol Methods 160(1):81-8). Vanadate in different concentrations was added to the cultures in addition to EGF (3 nM). It was shown that combining vanadate with EGF concentrations typically used for neurosphere cultures was not significantly altered by the addition of increasing concentrations of vanadate (FIG. 7).

Effects on Human Neural Stem Cells

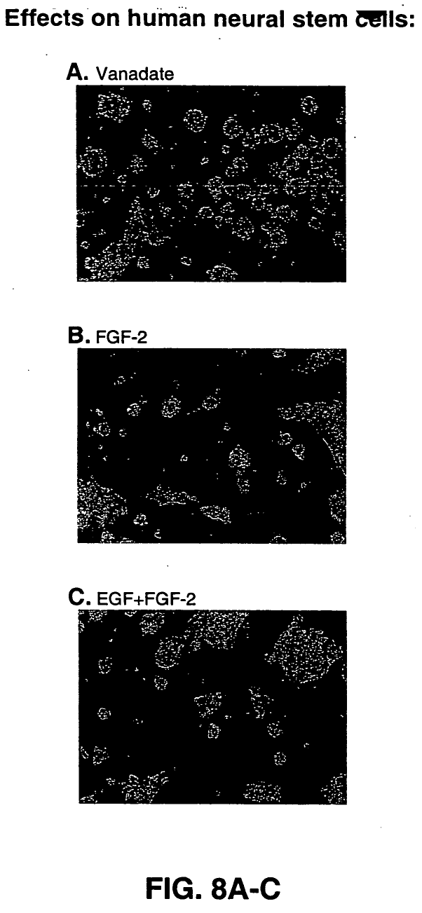

Cells plated in medium containing vanadate (10 uM) formed numerous spheres after 1 d, and virtually all spheres were free floating (FIG. 8a). FIGS. 8b and 8c show that with the addition of growth factors less sphere-like colonies formed and the majority of the spheres were adherent.

### CONCLUSION

We have discovered that a phosphate mimic when added to defined culture medium makes neural stem cells proliferate and form neurospheres in a fashion similar to treatment of the stem cells with protein growth factors (e.g., but not limited to EGF and FGF2). The mode of action of vanadate is most likely through, but not limited to, inhibition of PTPases thereby prolonging the action of insulin in the defined medium and of growth factor release by the cells to the surrounding medium.

The observed action of vanadate can be used in stem cell originating dysfunctions and diseases and as a tool to culture and maintain stem cells for the purpose of transplantation, drug screening and diagnosis of disease.

In conclusion, vanadate can be added to culture medium devoid of growth factors except for the supplement component insulin and thereby by itself trigger proliferation and expansion of adult derived neural stem cells. The effect is additive to two other proliferative cues (growth factor or cAMP). This effect can be used in vitro for, e.g., cell culture purposes or in vivo for therapeutic purposes.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

1. A method for culturing a stem cell, said method comprising propagating the stem cell in tissue culture medium comprising an agent selected from the group consisting of an inhibitor of a PTPase, a modulator of an enzyme with one or more phosphate binding sites, a phosphohydrolase, a pyrophosphatase, an alkaline phosphatase, an acid phosphatase, and a modulator of a protein with one or more pTyr recognition unit.

2. The method of claim 1, wherein the pTyr recognition unit is a SH2 domain.

3. The method of claim 1, wherein the enzyme with one or more phosphate binding sites is a glucose-6-phosphate dehydrogenase, a fructose-2,6-bisphosphatase, a phosphoglucomutase, a Mg²⁺ dependent ATPase, a plasma membrane Ca²⁺ ATPases, an endoplamic reticulum Ca²⁺-ATPases, a P-glycoprotein ATPase activity, a Mg²⁺-dependent vanadate-sensitive GS-conjugate export ATPase, a MRP/GS-X pump, and a Na⁺, K⁺-ATPase.

4. A method for culturing a stem cell, said method comprising propagating the stem cell in tissue culture medium comprising a phosphate mimic.

5. A method for culturing a stem cell, said method comprising incubating the stem cells in tissue culture medium comprising a phosphate mimic.

6. The method of claim 5, wherein the tissue culture medium comprises between 1 μM and 10 μM vanadate, between 10 μM and 50 μM vanadate, between 50 μM and 100 μM vanadate, between 100 μM and 500 μM vanadate or between 500 μM and 1000 μM vanadate.

7. The method of claim 5, wherein the tissue culture medium comprises a phosphate mimic, and wherein the culture medium is not supplemented with exogenously added growth factor.

8. The method of claim 5, wherein the tissue culture medium comprises a phosphate mimic, and wherein the incubating step elevates intracellular ATP levels in the neural stem cell by at least 25% compared to intracellular ATP levels in the stem cell incubated without phosphate mimic under otherwise same conditions.

9. (canceled)

10. A method for culturing a stem cell, said method comprising incubating the stem cell in tissue culture medium comprising a phosphate mimic, wherein the amount of phosphate mimic is sufficient for stem cells to exhibit at least 25% more proliferation than stem cells incubated without the phosphate mimic under otherwise same conditions.

11.-13. (canceled)

14. A method for culturing a neural stem cell, said method comprising incubating the neural stem cell in tissue culture medium comprising a phosphate mimic, wherein the incubating step increases formation of neurospheres from the neural stem cell by at least 25% compared to the formation of neurospheres from the stem cell incubated without phosphate mimic under otherwise same conditions.

15.-16. (canceled)

17. A cultured stem cell, wherein the cultured stem cell has been generated by the method of any one of claims 1, 4, 5, 10 or 14.

18. (canceled)

19. A method for culturing a progenitor cell, said method comprising propagating the progenitor cell in tissue culture medium comprising a phosphate mimic.

20. A method for culturing a progenitor cell, said method comprising incubating the progenitor cell in tissue culture medium comprising a phosphate mimic.

21. The method of claim 20, wherein the tissue culture medium comprises a phosphate mimic, and wherein the culture medium is not supplemented with exogenously added growth factor.

22. The method of claim 20, wherein the tissue culture medium comprises a phosphate mimic, and wherein the incubating step increases intracellular ATP levels in the progenitor cell by at least 25% compared to intracellular ATP levels in the progenitor cell incubated without phosphate mimic under otherwise same conditions.

23.-28. (canceled)

29. A cultured progenitor cell, wherein the cultured progenitor cell has been generated by the method of any one of claims 19 or 20.

30. The method of any one of claims 4, 5, 10, 14, 19, or 20, wherein the phosphate mimic is selected from the group consisting of a vanadium oxide, a derivative of a vanadium oxide, a polyoxometalate, a homopolyoxotungstate, a vanadium-substituted polyoxotungstate, an esterified derivative of 4-(fluoromethyl)phenyl phosphate, a homopolyoxoselenate, a vanadium-substituted polyoxoselenate, a homopolyoxomolybdate, a vanadium-substituted polyoxomolybdate, and a PTPase inhibitor.

31. The method of any one of claims 4, 5, 10, 14, 19, or 20, wherein the phosphate mimic is vanadate, orthovanadate, metavanadate, pervanadate, vanadate dimer, vanadate tetramer, vanadate pentamer, vanadate hexamer, vanadate heptamer, vanadate octamer, vanadate nonamer, vanadate decamer, vanadate polymer, vanadyl sulfate, bis(6, ethylpicolinato)(H(2)O)oxovanadium(IV) complex, bis(1-oxy-2-pyridinethiolato)oxovanadium(IV), bis(maltolato)oxovanadium (IV), bis(biguanidato)oxovanadium(IV), bis(N′N′-dimethylbiguanidato)oxovanadium(IV), bis(beta-phenethyl-biguanidato)oxovanadium(IV), peroxovanadate-nicotinic acid, aluminiofluoride, 4-(fluoromethyl)phenyl phosphate, tungstate, selenate, molybdate, Zn²⁺ or F⁻¹.

32. The method of any one of claims 31, wherein the phosphate mimic is vanadate.

33. The method of claim 32, wherein the concentration of vanadate in the culture medium is 1 μM, 10 μM, 50 μM, 100 μM, 500 μM or 1000 μM.

34. The method of any one of claims 1, 4, 5, 10, 14, 19, or 20, wherein the culture medium further comprises an agent selected from the group consisting of a growth factor, a Receptor Tyrosine Kinase agonist, and a growth factor secretagogue.

35. The method of any one of claims 1, 4, 5, 10, 14, 19, or 20, wherein the culture medium further comprises an agent selected from the group consisting of an agonist of cAMP accumulation, a Ca²⁺-transient triggering factor, and an agonist of cGMP accumulation.

36. (canceled)

37. The method of claim 4, 5, 10, 14, 19, or 20, wherein the cell does not substantially differentiate during the incubating step.

38.-43. (canceled)

44. The method of claim 5, wherein the stem cell undergoes self-renewal.

45. The method of claim 20, wherein the progenitor cell undergoes self-renewal.
